(19) United States
(12) Patent Application Publication
(10) Pub. No.: US 2012/0058198 A1
(43) Pub. Date: Mar. 8, 2012

(54) METHODS FOR TREATING AND PREVENTING PNEUMONIA AND VENTILATOR-ASSOCIATED TRACHEOBRONCHITIS

(75) Inventors: Robert W. Clarke, Medfield, MA (US); Richard Batycky, Newton, MA (US); Wesley H. Dehaan, Chelmsford, MA (US); David L. Hava, Natick, MA (US); Michael M. Lipp, Framingham, MA (US); John Hanrahan, West Roxbury, MA (US)

(73) Assignee: PULMATRIX, INC., Lexington, MA (US)

(21) Appl. No.: 13/259,615

(22) PCT Filed: Mar. 26, 2010
(86) PCT No.: PCT/US10/28901

§ 371 Date: Nov. 21, 2011

(30) Foreign Application Priority Data
Mar. 26, 2009 (US) 61163767
Jan. 25, 2010 (US) 61298092

(51) Int. Cl. A61P 31/04; A61K 33/06; A61K 33/14

(52) U.S. Cl. 424/602; 424/680; 424/682; 424/687; 514/557

(57) ABSTRACT

The invention relates to methods for treating bacterial infection of the respiratory tract, including pneumonias, such as ventilator-associated pneumonia, and to methods for treating ventilator-associated tracheobronchitis, comprising administering an effective amount of a salt formulation as an aerosol to the respiratory tract of an individual in need thereof. The formulations can also be used to reduce transmission of pathogen which can infect the respiratory tract, cause pneumonia or cause ventilator-associated tracheobronchitis.

### RELATED APPLICATIONS

This application claims the benefit of U.S. Application No. 61/298,092, filed Jan. 25, 2010, and U.S. Application No. 61/163,767, filed Mar. 26, 2009. The entire teachings of the above applications are incorporated herein by reference.

### BACKGROUND OF THE INVENTION

Pneumonia, a common disease caused by a great diversity of infectious agents, is responsible for enormous morbidity and mortality worldwide. Pneumonia is the third leading cause of death worldwide and the leading cause of death due to infectious disease in industrialized countries. In developing countries, approximately 2 million deaths (20% of all deaths) of children are due to pneumonia. Lancet Infect Dis., 2:25-32 (2002). The majority of patients with community-acquired pneumonia (CAP) in industrialized countries are treated as outpatients with a low mortality rate (usually less than 1%). For patients requiring inpatient management, the overall mortality rate increases up to approximately 12%.

In nosocomial pneumonia (hospital-acquired pneumonia, HAP; health-care associated pneumonia, HCAP) mortality increases substantially. HAP accounts for 15% of all nosocomial infections its mortality rate exceeds 30%, although the attributable mortality is lower. Am J Respir Crit Care Med., 157:1165-1172 (1998); Am J Med., 94:281-288 (1993); Chest., 119:373S-384S (2001). Requirement of mechanical ventilation is a high risk factor for the development of HAP with high mortality. This form of HAP, called ventilator-associated pneumonia (VAP) occurs in up to 47% of all intubated patients and varies among patient populations. Curr Opin Pulm Med., 11:236-241 (2005). VAP dramatically increases health care costs because it results in an increased length of stay in the hospital. Moreover, high mortality rates are reported that range from 34% in mixed medical/surgical intensive care unit patients to up to 57.1% in heart surgical patients. JAMA., 290:367-373 (2003); Crit Care Med., 31:1964-1970 (2003).

Bacteria are the most common cause of pneumonia in adults. Most CAP cases are due to infections with Streptococcus pneumoniae, Haemophilus influenzae, and Mycoplasma pneumoniae. Lancet., 362:1991-200 (2003); Curr Opin Pulm Med., 6:226-233 (2000). The majority of late onset-VAP cases is caused by Staphylococcus aureus, including antibiotic-resistant subtypes, Pseudomonas spp., Klebsiella spp., as well as Acitenobacter spp. Crit Care., 9:459-464 (2005).

Patients who are mechanically ventilated are also at risk for developing ventilator-associated tracheobronchitis (VAT). See, e.g., Tones et al., Critical Care, 9:255-256 (2005); Craven D., Critical Care, 12:157 (2008); Craven et al., Chest, 135:521-528 (2009). VAT, like VAP, is characterized by microbial colonization of the respiratory tract, and may progress to VAP. Craven et al., Chest, 135:521-528 (2009). VAT is associated with increased length of stay in intensive care units, and more days on mechanical ventilation. Craven D., Critical Care, 12:157 (2008). Clinical studies have shown that treating VAT with antibiotics reduces incidence of VAP, reduces the number of days on mechanical ventilation, and reduces mortality. Craven D., Critical Care, 12:157 (2008).

CAP and HAP represent an enormous economic burden to the public health systems. CAP alone causes costs of about US$ 20 billion in the United States due to more than 10 million visits to physicians, 64 million days of restricted activity and over 600,000 hospitalizations per year. Clin Infect Dis., 18:501-513 (1994); Am J Med., 78:45-51 (1985).

Increasing antimicrobial resistance of pathogens causing CAP (e.g. Streptococcus pneumoniae) and VAP (e.g. Pseudomonas aerugenosa, Staphylococcus aureus) as well as the increasing number of humans with increased susceptibility to pneumonia (e.g. geriatric and/or immunocompromised people) will aggravate the problem. Treat Respir Med., 4 Suppl 1:19-23.:19-23 (2005); Infection., 33:106-114 (2005); Curr Opin Pulm Med., 11:236-241 (2005).; Am J Respir Crit Care Med., 170:786-792 (2004); Curr Opin Pulm Med., 11:226-230 (2005). The development of new preventive and therapeutic strategies for pneumonia is urgently needed. A dire need exists for development of innovative therapeutic methods for treating pneumonia that are not limited to antibiotics.

### SUMMARY OF THE INVENTION

The invention relates to a method for treating pneumonia, comprising administering to an individual having pneumonia or exhibiting pneumonia-like symptoms, an effective amount of a formulation comprising a therapeutically effective amount of a calcium salt, wherein the formulation is administered as an aerosol to the respiratory tract (e.g., lung) of the individual.

The invention also relates to a method for reducing transmission of pathogens which cause pneumonia, comprising administering to an individual having pneumonia, exhibiting pneumonia-like symptoms, or at risk for infection by a pathogen that can cause pneumonia, an effective amount of a formulation comprising a therapeutically effective amount of a calcium salt, wherein the formulation is administered as an aerosol to the respiratory tract (e.g., lung) of the individual.

The invention further relates to a method of preventing pneumonia, comprising administering to an individual at risk for contracting pneumonia an effective amount of a formulation comprising a therapeutically effective amount of a calcium salt, wherein the formulation is administered as an aerosol to the respiratory tract (e.g., lung) of the individual.

The pneumonia is preferably bacterial pneumonia. For example, the bacterial pneumonia can be caused by Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia Burkholderia spp and combinations thereof. In some embodiments the pneumonia is community acquired pneumonia (CAP), ventilator associated pneumonia (VAP), hospital acquired pneumonia (HAP) or healthcare associated pneumonia (HCAP).

In a particular aspect, the invention relates to a method for treating ventilator-associated pneumonia (VAP), comprising administering to an individual having VAP an effective amount of a calcium salt formulation, wherein said calcium salt formulation is administered as an aerosol to the lung of said individual.

In another particular aspect, the invention relates to a method for prophylaxis of ventilator-associated pneumonia (VAP), comprising administering to an individual at risk for VAP, such as an intubated patient, an effective amount of a calcium salt formulation, wherein said calcium salt formulation is administered as an aerosol to the lung of said individual.

The invention relates to a method for treating VAT, comprising administering to an individual who has VAT or exhibits VAT-like symptoms, an effective amount of a formulation comprising a therapeutically effective amount of a calcium salt, wherein the formulation is administered as an aerosol to the respiratory tract (e.g., lung) of the individual.

The invention relates to a method for preventing VAT, comprising administering to an individual at risk for VAT, such as an intubated patient, an effective amount of a formulation comprising a therapeutically effective amount of a calcium salt, wherein the formulation is administered as an aerosol to the respiratory tract (e.g., lung) of the individual.

The invention relates to a method for treating (including prophylactically treating) a bacterial respiratory tract infection, comprising administering to an individual having a bacterial infection of the respiratory tract, exhibiting symptoms of a bacterial infection of the respiratory tract, or at risk of contracting a bacterial infection of the respiratory tract an effective amount of a calcium salt formulation, and an antibiotic agent. The invention also relates to a method for reducing transmission of a bacterial pathogen that causes a respiratory tract infection, comprising administering to an individual having a bacterial infection of the respiratory tract, exhibiting symptoms of a bacterial infection of the respiratory tract, or at risk of contracting a bacterial infection of the respiratory tract an effective amount of a calcium salt formulation, and an antibiotic agent.

The calcium salt can be calcium chloride, calcium carbonate, calcium acetate, calcium phosphate, calcium alginate, calcium stearate, calcium sorbate, calcium sulfate, calcium citrate, calcium lactate, calcium gluconate and the like and combinations thereof. In some embodiments, a calcium dose of about 0.001 mg/kg body weight to about 10 mg/kg body weight is administered to the respiratory tract (e.g., lungs). The formulation can be a liquid formulation or a dry powder.

In particular embodiments, the calcium salt formulation further comprises a sodium salt. The sodium salt can be sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium lactate, sodium borate, sodium gluconate, sodium metasilicate and the like and combinations thereof. In some embodiments, the ratio of calcium to sodium in the calcium salt formulation is about 8:1. In some embodiments, the sodium dose administered to the lungs is about 0.001 mg/kg body weight to about 10 mg/kg body weight.

The invention also relates to a salt formulation, as described herein, for use in therapy, and to the use of a salt formulation as described herein for the manufacture of a medicament for the treatment, prophylaxis and/or reduction in contagion of a disease described herein, such as a bacterial infection of the respiratory tract, pneumonia, VAP or VAT.

### BRIEF DESCRIPTION OF THE DRAWINGS

FIG. 1 is a schematic of a pass-through model used in the studies described herein.

FIG. 2 is a graph showing that calcium inhibits movement of K. pneumoniae across a mucus mimetic (sodium alginate) in a bacterial pass through assay. The mucus mimetic was exposed to 1.29% calcium chloride (0.12M) in 0.90% sodium chloride solution, or 0.90% sodium chloride and K. pneumoniae was added to the apical surface. The titer of bacteria in basolateral buffer was determined over time.

FIG. 3 is a graph showing that calcium inhibits movement of S. pneumoniae across a mucus mimetic (sodium alginate) in a bacterial pass through assay. The mucus mimetic was exposed to 1.29% calcium chloride (0.12M) in 0.90% sodium chloride solution, or 0.90% sodium chloride and S. pneumoniae was added to the apical surface. The titer of bacteria in basolateral buffer was determined over time.

FIG. 4 is a graph showing that magnesium reduces the movement of K. pneumoniae across a mucus mimetic (sodium alginate) in a bacterial pass through assay. The mucus mimetic was exposed to 0.12M magnesium chloride in 0.90% sodium chloride solution, or 0.90% sodium chloride and K. pneumoniae was added to the apical surface. The titer of bacteria in basolateral buffer was determined over time. Magnesium chloride inhibited movement across the mucus mimetic but to a lesser extent than calcium. (Compare to FIG. 2.)

FIG. 5 is a graph showing zinc and aluminum reduced the movement of K. pneumoniae across a mucus mimetic (sodium alginate) in a bacterial pass through assay. The mucus mimetic was exposed to 0.12M calcium chloride in 0.90% sodium chloride solution, 0.12M aluminum chloride in 0.90% sodium chloride solution, 0.12M zinc chloride in 0.90% sodium chloride solution, or 0.90% sodium chloride and K. pneumoniae was added to the apical surface. The titer of bacteria in basolateral buffer was determined over time. Zinc and aluminum inhibited movement across the mucus mimetic but to a lesser extent than calcium.

FIG. 6 is a graph showing prophylactic exposure of sodium alginate mimetic to calcium chloride inhibits the movement of K. pneumoniae across sodium alginate mucus mimetic. Bacteria were added 40 minutes before nebulization, immediately before nebulization, or 40 minutes after nebulization.

FIG. 7 is a graph showing calcium chloride inhibits the movement of bacteria through mucus mimetic in a dose dependent manner. The dose effect of calcium chloride is shown to reduce bacterial movement through mucus mimetic.

FIG. 8 is a graph showing calcium chloride alone, without 0.90% sodium chloride, inhibits the movement of bacteria through mucus mimetic in a dose dependent manner. The dose effect of calcium chloride is shown to reduce bacterial movement through mucus mimetic.

FIG. 9 is a graph showing reduced movement of P. aeruginosa across a mucus mimetic (sodium alginate) in a bacterial pass through assay. The mucus mimetic was exposed to 1.29% calcium chloride (0.12M) in 0.90% sodium chloride solution, or 0.90% sodium chloride, and P. aeruginosa was added to the apical surface. The titer of bacteria in basolateral buffer was determined over time.

FIG. 10A is a graph showing reduced movement of non-typeable Haemophilus influenzae (NHTI) across a mucus mimetic (sodium aliginate) in a bacterial pass through assay. The mucus mimetic was exposed to 0.12M calcium chloride in 0.90% sodium chloride solution, or 0.9% sodium chloride, and NHTI was added to the apical surface.

FIG. 10B is a graph showing reduced movement of S. aureus across a mucus mimetic (sodium aliginate) in a bacterial pass through assay. The mucus mimetic was exposed to 0.12M calcium chloride in 0.90% sodium chloride solution, or 0.9% sodium chloride, and S. aureus was added to the apical surface.

FIG. 11A is a schematic showing an in vitro simulated cough system. Bottled compressed air, filtered to remove particles >0.01 micrometers in diameter is used to fill the Pressurized Chamber to a set pressure to mimic the flow of a cough maneuver. To initiate a cough maneuver, the solenoid valve is actuated, releasing the compressed air through a pneumotachometer, which records the air flow rate, and a low resistance HEPA filter. Air enters the trough with airflow passing over the mucus mimetic and generating aerosol particles. The drip trap prevents any bulk motion of the mucus mimetic from entering the holding chamber while the generated aerosol enters the expandable holding chamber. After completion of the cough, the optical particle counter sizes and counts the aerosol particles in the holding chamber as it draws the air out of the chamber.

FIG. 11B is a graph showing calcium chloride is more effective than 0.90% saline in the suppression of bioparticle formation in an in vitro model. Mean (±SEM) cumulative particle counts were measured following simulated cough over mucus mimetic (MM) in a tracheal trough model (n=4 per condition). The effect of each test formulation was tested by topically treating the mimetic with nebulized aerosol prior to simulated cough and enumeration of the particles (0.3 to 25 μm) with an optical particle counter.

FIG. 11C is a graph showing suppresssion of pathogen containing bioparticle formation by exposure to 1.29% calcium chloride (0.12M) in 0.90% sodium chloride solution. Mucus mimetics were mixed with K. pneumoniae and added to the cough system. Following simulated cough, bioparticles were collected in liquid broth and the number of CFU determined. Mimetic treated with calcium aerosols reduced the number of partcles containing K. pneumoniae by 75% relative to the untreated control.

FIG. 12A is a graph showing that mice infected with S. pneumoniae and treated two hours after infection with CaCl₂-saline aerosol (1.29% calcium chloride (0.12M) in 0.90% sodium chloride) for fifteen minutes, have less bacterial burden than untreated controls. Each data point represents the data obtained from a single animal. The bar for each group represents the geometric mean of the group.

FIG. 12B is a graph showing that mice treated with CaCl₂-saline aerosol (1.29% calcium chloride (0.12M) in 0.90% sodium chloride) for fifteen minutes, two hours before infection with S. pneumoniae, have less bacterial burden than untreated controls. Each data point represents the data obtained from a single animal. The bar for each group represents the geometric mean of the group.

FIG. 13A is a graph showing that mice infected with S. pneumoniae and treated with MgCl₂-saline aerosol (0.12 M magnesium chloride in 0.90% sodium chloride) for fifteen minutes two hours before infection have a similar bacterial burden as untreated controls. Pooled data from multiple experiments are shown. Each data point represents the data obtained from a single animal. The bar for each group represents the geometric mean of the group. The data were statistically analyzed using a Mann-Whitney U test (ns=not significant).

FIG. 13B is a graph showing that mice infected with S. pneumoniae and pretreated with saline aerosol (0.90% sodium chloride) for fifteen minutes two hours before infection have a higher bacterial burden than animals pretreated with CaCl₂-saline aerosol (1.29% calcium chloride (0.12M) in 0.9% sodium chloride). Pooled data from multiple experiments are shown. Each data point represents the data obtained from a single animal. The bar for each group represents the geometric mean of the group. The data were statistically analyzed using a Mann-Whitney U test.

FIG. 14A shows that formulations comprising calcium chloride and sodium chloride (Ca²⁺:Na⁺ at 8:1 ratio) reduced lung bacterial burden. Mice were treated with the indicated formulations using a PariLC Sprint nebulizer and subsequently infected with S. pneumoniae. The lung bacterial burden in each animal is shown. Each circle represents data from a single animal and the bar depicts the geometric mean with the 95% confidence interval. Data for the NaCl, 0.5× and 1× groups are pooled from two or three independent experiments. Data from the 2× and 4× groups are from a single experiment.

FIG. 14B shows that increasing calcium dose with longer nebulization times did not significantly impact therapeutic efficacy. Mice were treated with saline (NaCl) or a calcium:sodium formulation (1×tonicity=isotonic; 8:1 Ca²⁺:Na⁺ at 8:1 molar ratio) using a Pari LC Sprint nebulizer and subsequently infected with S. pneumoniae. The lung bacterial burden in each animal is shown. Each circle represents data from a single animal and the bar depicts the geometric mean. Dosing times of 3 minutes or greater significantly reduced bacterial burdens relative to controls (one-way ANOVA; Tukey's multiple comparison post-test).

FIG. 14C is a graph showing the inhibition of bacterial infection by ampicillin, Formulation 10 (1×), saline, and Formulation 10 plus Ampicillin (Ampicillin+1×). The data were collected from three independent experiments (n=5-6 per group per experiment) and each experiment was normalized to the respective saline control. Each data point represents the percent of the untreated control for a single animal and the bar depicts the geometric mean plus or minus the 95% confidence interval. Groups of data were analyzed by Mann-Whitney U test. *** indicates p<0.001 compared to the saline control.

FIG. 15 is a graph showing that dry powder treatment reduced severity of bacterial pneumonia in a mouse model. Mice were treated with the indicated dry powder formulations and subsequently infected with S. pneumoniae. The lung bacterial burden in each animal is shown. Each circle represents data from a single animal and the bar depicts the geometric mean for the group. Data were normalized to the leucine control in each respective experiment. Data are pooled from two independent experiments. The treatment groups were compared to the leucine control group by two-tailed Student t-test.

### DETAILED DESCRIPTION OF THE INVENTION

The invention relates to methods for the treatment, prophylaxis and reduction in contagion of pneumonia and/or VAT. As described herein, the results of in vitro and in vivo studies into the treatment and prevention of pneumonia by administering salt formulations (e.g., formulations comprising a calcium salt, formulations comprising a calcium salt and a sodium salt) to the lungs showed that salt solutions can inhibit the ability of pathogens that cause pneumonia (e.g., S. pneumoniae, K. pneumoniae, P. arugenosa) to pass through mucus layers. This effect will reduce infection rates and is useful to treat or prevent pneumonia, because pathogens must pass through the airway lining fluid in order to establish infection and cause pneumonia or VAT. The studies described herein also demonstrate that administering salt formulations to the lungs of mice prior to or after infection with a pathogen that causes pneumonia or VAT lowered the pathogen burden in the mice, which is indicative of efficacy in treating and preventing pneumonia and/or VAT.

The term “pneumonia” is a term of art that refers to an inflammatory illness of the lung. Pneumonia can result from a variety of causes, including infection with bacteria, viruses, fungi, or parasites, and chemical or physical injury to the lungs. Typical symptoms associated with pneumonia include cough, chest pain, fever and difficulty breathing. Clinical diagnosis of pneumonia is well-known in the art and may include x-ray and/or examination of sputum.

The term “bacterial pneumonia” refers to pneumonia caused by bacterial infection, including for example, infection of the respiratory tract by Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof.

The term “viral pneumonia” refers to pneumonia caused by a viral infection. Viruses that commonly cause viral pneumonia include, for example, influenza virus, respiratory syncytial virus (RSV), adenovirus, and metapneumovirus. Herpes simplex virus is a rare cause of pneumonia for the general population, but is more common in newborns. People with weakened immune systems are also at risk for pneumonia caused by cytomegalovirus (CMV).

Pneumonias can be classified in several ways, including by the presence of anatomic changes in the lungs, microbiologic classification, radiological classification and combined clinical classification. Combined clinical classification combines factors such as age, risk factors for certain microorganisms, the presence of underlying lung disease and underlying systemic disease, and whether the person has been recently hospitalized. There are two broad categories of pneumonia: community-acquired pneumonia (CAP) and hospital-acquired pneumonia (HAP). A third category, healthcare-associated pneumonia (HCAP), occurs in patients living outside of the hospital who have recently been in close contact with the health care system, and lies between the two broad categories of pneumonia.

The term “community-acquired pneumonia (CAP)” as used herein refers to infectious pneumonia in a subject who has not recently been hospitalized. CAP is the most common type of pneumonia. S. pneumoniae is the most common pathogen that causes community-acquired pneumonia worldwide. CAP is the fourth most common cause of death in the United Kingdom and the sixth most common cause of death in the United States.

The term “hospital-acquired pneumonia (HAP)” as used herein refers to pneumonia acquired during or after hospitalization for another illness or procedure with onset at least 72 hours after admission to the hospital. Hospitalized patients may have many risk factors for pneumonia, including mechanical ventilation, prolonged malnutrition, underlying heart and lung diseases, decreased amounts of stomach acid, and immune disturbances. Additionally, the microorganisms present in a hospital are an additional risk factor. Hospital-acquired microorganisms may include drug resistant bacteria such as methicilllin resistant Staphylococcus aureas (MRSA), Pseudomonas spp., Enterobacter spp., and Serratia spp.. “Ventilator-associated pneumonia (VAP)” is a subset of HAP. As used herein, “ventilator-associated pneumonia (VAP)” is pneumonia which occurs after at least 48 hours of intubation or mechanical ventilation.

The term “ventilator-associated tracheobronchitis” (VAP) is a term of art that refers to a spectrum of disease that occurs in intubated patients, and affected patients usually show clinical signs of lower respiratory tract infection. See, e.g., Craven et al., Chest, 135:521-528 (2009). VAT is characterized by microbiological colonization of the respiratory tract, and common pathogens for VAT include Pseudomonas aeruginosa, Acinobacter baumannii, and methicillin-resistant Staphylococcus aureus.

The term “aerosol” as used herein refers to any preparation of a fine mist of particles (including liquid and non-liquid particles, e.g., dry powders), typically with a volume median geometric diameter of about 0.1 to about 30 microns or a mass median aerodynamic diameter of between about 0.5 and about 10 microns. Preferably the volume median geometric diameter for the aerosol particles is less than about 10 microns. The preferred volume median geometric diameter for aerosol particles is about 5 microns. For example, the aerosol can contain particles that have a volume median geometric diameter between about 0.1 and about 30 microns, between about 0.5 and about 20 microns, between about 0.5 and about 10 microns, between about 1.0 and about 3.0 microns, between about 1.0 and 5.0 microns, between about 1.0 and 10.0 microns, between about 5.0 and 15.0 microns. Preferably the mass median aerodynamic diameter is between about 0.5 and about 10 microns, between about 1.0 and about 3.0 microns, or between about 1.0 and 5.0 microns.

The term “respiratory tract” as used herein includes the upper respiratory tract (e.g., nasal passages, nasal cavity, throat, pharynx), respiratory airways (e.g., larynx, tranchea, bronchi, bronchioles) and lungs (e.g., respiratory bronchioles, alveolar ducts, alveolar sacs, alveoli).

As used herein, “1×” tonicity refers to a solution that is isotonic relative to normal human blood and cells. Solutions that are hypotonic or hypertonic in comparison to normal human blood and cells are described relative to a 1× solution using an appropriate multiplier. For example, a hypotonic solution may have 0.1×, 0.25× or 0.5× tonicity, and a hypertonic solution may have 2×, 3×, 4×, 5×, 6×, 7×, 8×, 9× or 10× tonicity.

The term “dry powder” as used herein refers to a composition contains finely dispersed respirable dry particles that are capable of being dispersed in an inhalation device and subsequently inhaled by a subject. Such dry powder or dry particle may contain up to about 15% water or other solvent, or be substantially free of water or other solvent, or be anhydrous.

The invention described herein provides methods for treating, preventing or reducing contagion of pneumonia (e.g., CAP, HAP, HCAP, VAP) and/or VAT that comprises administering salt formulations to the respiratory tract (e.g., pulmonary administration). Pulmonary delivery of salt formulations provides a safe, low-cost medical intervention that is suitable to treat, prevent or reduce contagion of pneumonia or VAT caused by a variety of pathogens (e.g., S. pneumoniae, K. pneumoniae, P. arugenosa).

### Formulations

Salt formulations (e.g., calcium salt formulations) for use in the methods described herein contain at least one salt as an active ingredient, and can optionally contain additional salts or agents. Without wishing to be bound by a particular theory, it is believed that therapeutic and prophylactic benefits produced by the salt formulations and the methods described herein, result from an increase in the amount of cation (cation from the salt, such as Ca²⁺) in the respiratory tract, e.g., in the lung mucus or airway lining fluid after administration of the salt formulation.

The salt formulations can include any salt form of the elements sodium, potassium, magnesium, calcium, aluminum, silicon, scandium, titanium, vanadium, chromium, cobalt, nickel, copper, manganese, zinc, tin, silver and similar elements, that is non-toxic when administered to the respiratory tract. The salt formulation can be in any desired form, such as a solution, emulsion, suspension, or a dry powder. Preferred salt formulations, such as solutions and dry powders, can be aerosolized. Preferred salt formulations contain sodium salts (e.g., saline (0.15 M NaCl or 0.90% solution)), calcium salts, or mixtures of sodium salts and calcium salts. When the formulation comprises a calcium salt, a sodium salt or a combination of a calcium salt or a sodium salt, it can, if desired, also contain one or more other salts. The salt formulations can comprise multiple doses or be a unit dose composition as desired.

Suitable sodium salts include, for example, sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium lactate, sodium borate, sodium gluconate, sodium metasilicate, and the like, or a combination thereof.

Suitable calcium salts include, for example, calcium chloride, calcium carbonate, calcium acetate, calcium phosphate, calcium alginate, calcium stearate, calcium sorbate, calcium sulfate, calcium gluconate, calcium citrate, calcium lactate, and the like, or a combination thereof.

Suitable magnesium salts include, for example, magnesium chloride, magnesium carbonate, magnesium acetate, magnesium phosphate, magnesium aliginate, magnesium sulfate, magnesium stearate, magnesium sorbate, magnesium gluconate, magnesium citrate, magnesium lactate, magnesium trisilicate, magnesium chloride, and the like, or a combination thereof.

Suitable potassium salts include, for example, potassium bicarbonate, potassium chloride, potassium citrate, potassium borate, potassium bisulfite, potassium biphosphate, potassium alginate, potassium benzoate, and the like. Additional suitable salts include cupric sulfate, chromium chloride, stannous chloride, and similar salts. Other suitable salts include zinc chloride, aluminum chloride and silver chloride.

The salt formulation is generally prepared in or comprises a physiologically acceptable carrier or excipient. For salt formulations in the form of solutions, suspensions or emulsions, any suitable carrier or excipient can be included. Suitable carriers include, for example, aqueous, alcoholic/aqueous, and alcohol solutions, emulsions or suspensions, including water, saline, ethanol/water solution, ethanol solution, buffered media, propellants and the like. For salt formulations in the form of dry powders, suitable carrier or excipients include, for example, sugars (e.g., lactose, trehalose), sugar alcohols (e.g., mannitol, xylitol, sorbitol), amino acids (e.g., glycine, alanine, leucine, isoleucine), dipalmitoylphosphosphatidylcholine (DPPC), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, alkylated sugars, sodium phosphate, maltodextrin, human serum albumin (e.g., recombinant human serum albumin), biodegradable polymers (e.g., PLGA), dextran, dextrin, and the like. If desired, the salt formulations can also contain additives, preservatives, or fluid, nutrient or electrolyte replenishers (See, generally, Remington's Pharmaceutical Sciences, 17th Edition, Mack Publishing Co., PA, 1985).

The salt formulation preferably contains a concentration of salt (e.g., calcium salt, sodium salt) that permits convenient administration of an effective amount of the formulation to the respiratory tract. For example, it is generally desirable that liquid formulations not be so dilute so as to require a large amount of the formulation to be nebulized in order to deliver an effective amount to the respiratory tract of a subject. Long administration periods are disfavored, and generally the formulation should be concentrated enough to permit an effective amount to be administered to the respiratory tract (e.g., by inhalation of aerosolized formulation, such as nebulized liquid or aerosolized dry powder) or nasal cavity in no more than about 120 minutes, no more than about 90 minutes, no more than about 60 minutes, no more than about 45 minutes, no more than about 30 minutes, no more than about 25 minutes, no more than about 20 minutes, no more than about 15 minutes, no more than about 10 minutes, no more than about 7.5 minutes, no more than about 5 minutes, no more than about 4 minutes, no more than about 3 minutes, no more than about 2 minutes, no more than about 1 minute, no more than about 45 seconds, or no more than about 30 seconds. For example, a liquid salt formulation (e.g., a calcium salt formulation) can contain about 0.01% to about 30% salt (w/v), between 0.1% to about 20% salt (w/v), between 0.1% to about 10% salt (w/v). Liquid formulations can contain about 0.001M to about 1.5M salt, about 0.01M to about 1.0M salt, about 0.01M to about 0.90M salt, about 0.01M to about 0.8M salt, about 0.01M to about 0.7M salt, about 0.01M to about 0.6M salt, about 0.01M to about 0.5M salt, about 0.01M to about 0.4M salt, about 0.01M to about 0.3M salt, about 0.01M to about 0.2M salt, about 0.1M to about 1.0M salt, about 0.1M to about 0.90M salt, about 0.1M to about 0.8M salt, about 0.1 M to about 0.7M salt, about 0.1 M to about 0.6M salt, about 0.1 M to about 0.5M salt, about 0.1M to about 0.4M salt, about 0.1M to about 0.3M salt, or about 0.1M to about 0.2M salt.

In further examples, a liquid salt formulation may contain from about 0.115 M to 1.15 M Ca²⁺ ion, from about 0.116 M to 1.15 M Ca²⁺ ion, from about 0.23 M to 1.15 M Ca²⁺ ion, from about 0.345 M to 1.15 M Ca²⁺ ion, from about 0.424 M to 1.15 M Ca²⁺ ion, from about 0.46 M to 1.15 M Ca²⁺ ion, from about 0.575 M to 1.15 M Ca²⁺ ion, from about 0.69 M to 1.15 M Ca²⁺ ion, from about 0.805 M to 1.15 M Ca²⁺ ion, from about 0.849 M to 1.15 M Ca²⁺ ion, or from about 1.035 M to 1.15 M Ca²⁺ ion. The solubility of certain calcium salts (e.g., calcium carbonate, calcium citrate) can limit the preparation of solutions. In such situations, the liquid formulation may be in the form of a suspension that contains the equivalent amount of calcium salt that would be needed to achieve the desired molar concentration.

When the salt formulation contains a sodium salt, such as a formulation that contains a calcium salt and a sodium salt, the Na⁺ ion in a liquid pharmaceutical formulation can be dependent upon the desired Ca²⁺:Na⁺ ratio. For example, the liquid formulation may contain from about 0.053 M to 0.3 M Na⁺ ion, from about 0.075 M to 0.3 M Na⁺ ion, from about 0.106 M to 0.3 M Na⁺ ion, from about 0.15 M to 0.3 M Na⁺ ion, from about 0.225 M to 0.3 M Na⁺ ion, from about 0.008 M to 0.3 M Na⁺ ion, from about 0.015 M to 0.3 M Na⁺ ion, from about 0.016 M to 0.3 M Na⁺ ion, from about 0.03 M to 0.3 M Na⁺ ion, from about 0.04 M to 0.3 M Na⁺ ion, from about 0.08 M to 0.3 M Na⁺ ion, from about 0.01875 M to 0.3 M Na⁺ ion, from about 0.0375 M to 0.3 M Na⁺ ion, from about 0.075 M to 0.6 M Na⁺ ion, from about 0.015 M to 0.6 M Na⁺ ion, or from about 0.3 M to 0.6 M Na⁺ ion.

Dry powder formulations can contain at least about 10% salt by weight, at least about 20% salt by weight, at least about 30% salt by weight, at least about 40% salt by weight, at least about 50% salt by weight, at least about 60% salt by weight, at least about 70% salt by weight, at least about 75% salt by weight, at least about 80% salt by weight, at least about 85% salt by weight, at least about 90% salt by weight, at least about 95% salt by weight, at least about 96% salt by weight, at least about 97% salt by weight, at least about 98% salt by weight, or at least about 99% salt by weight. For example, some dry powder formulations contain about 20% to about 80% salt by weight, about 20% to about 70% salt by weight, about 20% to about 60% salt by weight, or can consist substantially of salt(s).

Preferred salt formulations contain a calcium salt. Certain calcium salts provide two or more moles of Ca²⁺ per mole of calcium salt upon dissolution. Such calcium salts may be particularly suitable to produce liquid or dry powder formulations that are dense in calcium, and therefore, can deliver an effective amount of cation (e.g., Ca²⁺, Na⁺, or Ca²⁺ and Na⁺). For example, one mole of calcium citrate provides three moles of Ca²⁺ upon dissolution. It is also generally preferred that the calcium salt is a salt with a low molecular weight and/or contain low molecular weight anions. Low molecular weight calcium salts, such as calcium salts that contain calcium ions and low molecular weight anions, are calcium dense relative to high molecular salts and calcium salts that contain high molecular weight anions. It is generally preferred that the calcium salt has a molecular weight of less than about 1000 g/mol, less than about 950 g/mol, less than about 900 g/mol, less than about 850 g/mol, less than about 800 g/mol, less than about 750 g/mol, less than about 700 g/mol, less than about 650 g/mol, less than about 600 g/mol, less than about 550 g/mol, less than about 510 g/mol, less than about 500 g/mol, less than about 450 g/mol, less than about 400 g/mol, less than about 350 g/mol, less than about 300 g/mol, less than about 250 g/mol, less than about 200 g/mol, less than about 150 g/mol, less than about 125 g/mol, or less than about 100 g/mol. In addition or alternatively, it is generally preferred that the calcium ion contributes a substantial portion of the weight to the overall weight of the calcium salt. It is generally preferred that the calcium ion weigh at least 10% of the overall calcium salt, at least 16%, at least 20%, at least 24.5%, at least 26%, at least 31%, at least 35%, or at least 38% of the overall calcium salt.

Some salt formulations contain a calcium salt in which the weight ratio of calcium to the overall weight of said calcium salt is between about 0.1 to about 0.5. For example, the weight ratio of calcium to the overall weight of said calcium salt is between about 0.15 to about 0.5, between about 0.18 to about 0.5, between about 0.2 to about 5, between about 0.25 to about 0.5, between about 0.27 to about 0.5, between about 0.3 to about 5, between about 0.35 to about 0.5, between about 0.37 to about 0.5, or between about 0.4 to about 0.5.

Some salt formulations contain a calcium salt and a sodium salt, for example 0.12 M calcium chloride in 0.15 M sodium chloride, or 1.3% (w/v) calcium chloride in 0.90% saline. Some salt formulations that contain a calcium salt and a sodium salt are characterized by the ratio of calcium:sodium (mole:mole). Suitable ratios of calcium:sodium (mole:mole) can range from about 0.1:1 to about 32:1, about 0.5:1 to about 16:1, or about 1:1 to about 8:1. For example, the ratio of calcium:sodium (mole:mole) can be about 0.77:1, about 1:1, about 1:1.3, about 1:2, about 4:1, about 8:1, or about 16:1. In particular examples, the salt formulations contain calcium chloride and sodium chloride, and have a calcium:sodium ratio of about 8:1 (mole:mole).

In certain aspects, the salt formulation that contains a calcium salt and a sodium salt and the ratio of Ca⁺² to Na⁺ is from about 4:1 (mole:mole) to about 16:1 (mole:mole). For example, the formulations can contain a ratio of Ca⁺² to Na⁺ from about 5:1 (mole:mole) to about 16:1 (mole:mole), from about 6:1 (mole:mole) to about 16:1 (mole:mole), from about 7:1 (mole:mole) to about 16:1 (mole:mole), from about 8:1 (mole:mole) to about 16:1 (mole:mole), from about 9:1 (mole:mole) to about 16:1 (mole:mole), from about 10:1 (mole:mole) to about 16:1 (mole:mole), from about 11:1 (mole:mole) to about 16:1 (mole:mole), from about 12:1 (mole:mole) to about 16:1 (mole:mole), from about 13:1 (mole:mole) to about 16:1 (mole:mole), from about 14:1 (mole:mole) to about 16:1 (mole:mole), from about 15:1 (mole:mole) to about 16:1 (mole:mole).

In certain aspects, the salt formulation contains a calcium salt and a sodium salt and the ratio of Ca⁺² to Na⁺ is from about 4:1 (mole:mole) to about 8:1 (mole:mole). For example, the formulations can contain a ratio of Ca⁺² to Na⁺ from about 5:1 (mole:mole) to about 8:1 (mole:mole), from about 6:1 (mole:mole) to about 8:1 (mole:mole), from about 7:1 (mole:mole) to about 8:1 (mole:mole).

In certain aspects, the salt formulation contains a calcium salt and a sodium salt and the ratio of Ca⁺² to Na⁺ is from about 4:1 (mole:mole) to about 5:1 (mole:mole), from about 4:1 (mole:mole) to about 6:1 (mole:mole), from about 4:1 (mole:mole) to about 7:1 (mole:mole), from about 4:1 (mole:mole) to about 8:1 (mole:mole), from about 4:1 (mole:mole) to about 9:1 (mole:mole), from about 4:1 (mole:mole) to about 10:1 (mole:mole), from about 4:1 (mole:mole) to about 11:1 (mole:mole), from about 4:1 (mole:mole) to about 12:1 (mole:mole), from about 4:1 (mole:mole) to about 13:1 (mole:mole), from about 4:1 (mole:mole) to about 14:1 (mole:mole), from about 4:1 (mole:mole) to about 15:1 (mole:mole).

The salt formulations can contain a ratio of Ca⁺² to Na⁺ from about 4:1 (mole:mole) to about 12:1 (mole:mole), from about 5:1 (mole:mole) to about 11:1 (mole:mole), from about 6:1 (mole:mole) to about 10:1 (mole:mole), from about 7:1 (mole:mole) to about 9:1 (mole:mole).

In particular examples, the ratio of Ca⁺² to Na⁺ is about 4:1 (mole:mole), about 4.5:1 (mole:mole), about 5:1 (mole:mole), about 5.5:1 (mole:mole), about 6:1 (mole:mole), about 6.5:1 (mole:mole), 7:1 (mole:mole), about 7.5:1 (mole:mole), about 8:1 (mole:mole), about 8.5:1 (mole:mole), about 9:1 (mole:mole), about 9.5:1 (mole:mole), about 10:1 (mole:mole), about 10.5:1 (mole:mole), about 11:1 (mole:mole), about 11.5:1 (mole:mole), about 12:1 (mole:mole), about 12.5:1 (mole:mole), about 13:1 (mole:mole), about 13.5:1 (mole:mole), about 14:1 (mole:mole), about 14.5:1 (mole:mole), about 15:1 (mole:mole), about 15.5:1 (mole:mole), or about 16:1 (mole:mole).

In more particular examples, the ratio of Ca⁺² to Na⁺ is about 8:1 (mole:mole) or about 16:1 (mole:mole).

Aqueous liquid salt formulations of this type can vary in tonicity and in the concentrations of calcium salt and sodium salt that are present in the formulation. For example, the salt formulation can contain 0.053 M CaCl₂ and 0.007 M NaCl (0.59% CaCl₂, 0.04% NaCl) and be hypotonic, 0.106 M CaCl₂ and 0.013 M NaCl (1.18% CaCl₂, 0.08% NaCl) and be isotonic, 0.212 M CaCl₂ and 0.027 M NaCl (2.35% CaCl₂, 0.027% NaCl) and be hypertonic, 0.424 M CaCl₂ and 0.054 M NaCl (4.70% CaCl₂, 0.054% NaCl) and be hypertonic, or 0.849 M CaCl₂ and 0.106 M NaCl (9.42% CaCl₂, 0.62% NaCl) and be hypertonic.

The salt formulation can be hypotonic, isotonic or hypertonic as desired. For example, any of the salt formulations described herein may have about 0.1× tonicity, about 0.25× tonicity, about 0.5× tonicity, about 1× tonicity, about 2× tonicity, about 3× tonicity, about 4× tonicity, about 5× tonicity, about 6× tonicity, about 7× tonicity, about 8× tonicity, about 9× tonicity, about 10× tonicity, at least about 1× tonicity, at least about 2× tonicity, at least about 3× tonicity, at least about 4× tonicity, at least about 5× tonicity, at least about 6× tonicity, at least about 7× tonicity, at least about 8× tonicity, at least about 9× tonicity, at least about 10× tonicity, between about 0.1× to about 1×, between about 0.1× to about 0.5×, between about 0.5× to about 2×, between about 1× to about 4×, between about 1× to about 2×, between about 2× to about 10×, or between about 4× to about 8×.

If desired, the salt formulation can include one or more additional agents, such as mucoactive or mucolytic agents, surfactants, antibiotics, antivirals, antihistamines, cough suppressants, bronchodilators, anti-inflammatory agents, steroids, vaccines, adjuvants, expectorants, macromolecules, therapeutics that are helpful for chronic maintenance of CF.

Examples of suitable mucoactive or mucolytic agents include MUC5AC and MUC5B mucins, DNA-ase, N-acetylcysteine (NAC), cysteine, nacystelyn, dornase alfa, gelsolin, heparin, heparin sulfate, P2Y2 agonists (e.g. UTP, INS365), hypertonic saline, and mannitol.

Suitable surfactants include L-alpha-phosphatidylcholine dipalmitoyl (“DPPC”), diphosphatidyl glycerol (DPPG), 1,2-Dipalmitoyl-sn-glycero-3-phospho-L-serine (DPPS), 1,2-Dipalmitoyl-sn-glycero-3-phosphocholine (DSPC), 1,2-Distearoyl-sn-glycero-3-phosphoethanolamine (DSPE), 1-palmitoyl-2-oleoylphosphatidylcholine (POPC), fatty alcohols, polyoxyethylene-9-lauryl ether, surface active fatty, acids, sorbitan trioleate (Span 85), glycocholate, surfactin, poloxomers, sorbitan fatty acid esters, tyloxapol, phospholipids, and alkylated sugars.

If desired, the salt formulation can contain an antibiotic. For example, salt formulations for treating bacterial pneumonia or VAT, can further comprise an antibiotic, such as a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), tuberculosis-mycobacterium antibiotics and the like.

If desired, the salt formulation can contain an agent for treating infections with mycobacteria, such as Mycobacterium tuberculosis. Suitable agents for treating infections with mycobacteria (e.g., M. tuberculosis) include an aminoglycoside (e.g. capreomycin, kanamycin, streptomycin), a fluoroquinolone (e.g. ciprofloxacin, levofloxacin, moxifloxacin), isozianid and isozianid analogs (e.g. ethionamide), aminosalicylate, cycloserine, diarylquinoline, ethambutol, pyrazinamide, protionamide, rifampin, and the like.

If desired, the salt formulation can contain a suitable antiviral agent, such as oseltamivir, zanamavir amantidine or rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, and acyclovir. Salt formulation can contain a suitable anti-influenza agent, such as zanamivir, oseltamivir, amantadine, or rimantadine.

Suitable antihistamines include clemastine, asalastine, loratadine, fexofenadine and the like.

Suitable cough suppressants include benzonatate, benproperine, clobutinal, diphenhydramine, dextromethorphan, dibunate, fedrilate, glaucine, oxalamine, piperidione, opiods such as codine and the like.

Suitable brochodilators include short-acting beta₂ agonists, long-acting beta₂ agonists (LABA), long-acting muscarinic anagonists (LAMA), combinations of LABAs and LAMAs, methylxanthines, and the like. Suitable short-active beta₂ agonists include albuterol, epinephrine, pirbuterol, levalbuterol, metaproteronol, maxair, and the like. Suitable LABAs include salmeterol, formoterol and isomers (e.g. arformoterol), clenbuterol, tulobuterol, vilanterol (Revolair™), indacaterol, and the like. Examples of LAMAs include tiotroprium, glycopyrrolate, aclidinium, ipratropium and the like. Examples of combinations of LABAs and LAMAs include indacaterol with glycopyrrolate, indacaterol with tiotropium, and the like. Examples of methylxanthine include theophylline, and the like.

Suitable anti-inflammatory agents include leukotriene inhibitors, PDE4 inhibitors, other anti-inflammatory agents, and the like. Suitable leukotriene inhibitors include montelukast (cystinyl leukotriene inhibitors), masilukast, zafirleukast (leukotriene D4 and E4 receptor inhibitors), zileuton (5-lipoxygenase inhibitors), and the like. Suitable PDE4 inhibitors include cilomilast, roflumilast, and the like. Other anti-inflammatory agents include omalizumab (anti IgE immunoglobulin), IL-13 and IL-13 receptor inhibitors (such as AMG-317, MILR1444A, CAT-354, QAX576, IMA-638, Anrukinzumab, IMA-026, MK-6105,DOM-0910 and the like), IL-4 and IL-4 receptor inhibitors (such as Pitrakinra, AER-003,AIR-645, APG-201, DOM-0919 and the like), IL-1 inhibitors such as canakinumab, CRTh2 receptor antagonists such as AZD1981 (from AstraZeneca), neutrophil elastase inhibitor such as AZD9668 (from AstraZeneca), P38 kinase inhibitor such as losmapimed, and the like.

Suitable steroids include corticosteroids, combinations of corticosteroids and LABAs, combinations of corticosteroids and LAMAs, and the like. Suitable corticosteroids include budesonide, fluticasone, flunisolide, triamcinolone, beclomethasone, mometasone, ciclesonide, dexamethasone, and the like. Combinations of corticosteroids and LABAs include salmeterol with fluticasone, formoterol with budesonide, formoterol with fluticasone, formoterol with mometasone, indacaterol with mometasone, and the like.

Suitable expectorants include guaifenesin, guaiacolculfonate, ammonium chloride, potassium iodide, tyloxapol, antimony pentasulfide and the like.

Suitable vaccines such as nasally inhaled influenza vaccines and the like.

Suitable macromolecules include proteins and large peptides, polysaccharides and oligosaccharides, and DNA and RNA nucleic acid molecules and their analogs having therapeutic, prophylactic or diagnostic activities. Proteins can include antibodies such as monoclonal antibody. Nucleic acid molecules include genes, antisense molecules such as SiRNAs that bind to complementary DNA, RNA, or ribosomes to inhibit transcription or translation.

Selected therapeutics that are helpful for chronic maintenance of CF include antibiotics/macrolide antibiotics, bronchodilators, inhaled LABAs, and agents to promote airway secretion clearance. Suitable examples of antibiotics/macrolide antibiotics include tobramycin, azithromycin, ciprofloxacin, colistin, and the like. Suitable examples of bronchodilators include inhaled short-acting beta₂ agonists such as albuterol, and the like. Suitable examples of inhaled LABAs include salmeterol, formoterol, and the like. Suitable examples of agents to promote airway secretion clearance include dornase alfa, hypertonic saline, and the like.

Dry powder formulations are prepared with the appropriate particle diameter, surface roughness, and tap density for localized delivery to selected regions of the respiratory tract. For example, higher density or larger particles may be used for upper airway delivery. Similarly, a mixture of different sized particles can be administered to target different regions of the lung in one administration.

As used herein, the phrase “aerodynamically light particles” refers to particles having a tap density less than about 0.4 g/cm³. The tap density of particles of a dry powder may be obtained by the standard USP tap density measurement. Tap density is a common measure of the envelope mass density. The envelope mass density of an isotropic particle is defined as the mass of the particle divided by the minimum sphere envelope volume in which it can be enclosed. Features contributing to low tap density include irregular surface texture and porous structure.

Dry powder formulations (“DPFs”) with large particle size have improved flowability characteristics, such as less aggregation (Visser, J., Powder Technology 58: 1-10 (1989)), easier aerosolization, and potentially less phagocytosis. Rudt, S. and R. H. Muller. J. Controlled Release, 22: 263-272 (1992); Tabata Y., and Y. Ikada. J. Biomed. Mater. Res. 22: 837-858 (1988). Dry powder aerosols for inhalation therapy are generally produced with mass median aerodynamic diameters primarily in the range of less than 5 microns, although dry powders that have any desired range in aerodynamic diameter can be produced. Ganderton D., J. Biopharmaceutical Sciences, 3:101-105 (1992); Gonda, I. “Plysico-Chemical Principles in Aerosol Delivery.” in Topics in Pharmaceutical Sciences 1991, Crommelin, D. J. and K. K. Midha, Eds., Medpharm Scientific Publishers, Stuttgart, pp. 95-115 (1992). Large “carrier” particles (containing no salt formulation) can be co-delivered with therapeutic aerosols to aid in achieving efficient aerosolization among other possible benefits. French, D. L., Edwards, D. A. and Niven, R. W., J. Aerosol Sci. 27: 769-783 (1996). Particles with degradation and release times ranging from seconds to months can be designed and fabricated by established methods in the art.

Generally, salt formulations that are dry powders may be produced by spray drying, freeze drying, jet milling, single and double emulsion solvent evaporation, and supercritical fluids. Preferably, salt formulations are produced by spray drying, which entails preparing a solution containing the salt and other components of the formulation, spraying the solution into a closed chamber, and removing the solvent with a heated gas stream.

Spray dried powders that contain salts with sufficient solubility in water or aqueous solvents, such as calcium chloride and calcium lactate, can be readily prepared using conventional methods. Some salts, such as calcium citrate and calcium carbonate, have low solubility in water and other aqueous solvents. Spray dried powders that contain such salts can be prepared using any suitable method. One suitable method involves combining other more soluble salts in solution and permitting reaction (precipitation reaction) to produce the desired salt for the dry powder formulation. For example, if a dry powder formulation comprising calcium citrate and sodium chloride is desired, a solution containing the high solubility salts calcium chloride and sodium citrate can be prepared. The precipitation reaction leading to calcium citrate is 3 CaCl₂+2 Na₃Cit→Ca₃Cit₂+6 NaCl. It is preferable that the sodium salt is fully dissolved before the calcium salt is added and that the solution is continuously stirred. The precipitation reaction can be allowed to go to completion or stopped before completion, e.g., by spray drying the solution, as desired. The resulting solution may appear clear with fully dissolved salts or a precipitate may form. Depending on reaction conditions, a precipitate may form quickly or over time. Solutions that contain a light precipitate, or even slurries, that result in formation of a stable homogenous suspension can be spray dried.

Alternatively, two saturated or sub-saturated solutions are fed into a static mixer in order to obtain a saturated or supersaturated solution post-static mixing. Preferably, the post-spray drying solution is supersaturated. The two solutions may be aqueous or organic, but are preferably substantially aqueous. The post-static mixing solution is then fed into the atomizing unit of a spray dryer. In a preferable embodiment, the post-static mixing solution is immediately fed into the atomizer unit. Some examples of an atomizer unit include a two-fluid nozzle, a rotary atomizer, or a pressure nozzle. Preferably, the atomizer unit is a two-fluid nozzle. In one embodiment, the two-fluid nozzle is an internally mixing nozzle, meaning that the gas impinges on the liquid feed before exiting to the most outward orifice. In another embodiment, the two-fluid nozzle is an externally mixing nozzle, meaning that the gas impinges on the liquid feed after exiting the most outward orifice.

Dry powder formulations can also be prepared by blending individual components into the final formulation. For example, a first dry powder that contains a calcium salt can be blended with a second dry powder that contains a sodium salt to produce a dry powder salt formulation that contains a calcium salt and a sodium salt. If desired, additional dry powders that contain excipients (e.g., lactose) and/or other active ingredients (e.g., antibiotic, antiviral) can be included in the blend. The blend can contain any desired relative amounts or ratios of salts, excipients and other ingredients (e.g., antibiotics, antivirals).

If desired, dry powders can be prepared using polymers, that are tailored to optimize particle characteristics including: i) interactions between the agent (e.g., salt) to be delivered and the polymer to provide stabilization of the agent and retention of activity upon delivery; ii) rate of polymer degradation and thus agent release profile; iii) surface characteristics and targeting capabilities via chemical modification; and iv) particle porosity. Polymeric particles may be prepared using single and double emulsion solvent evaporation, spray drying, solvent extraction, solvent evaporation, phase separation, simple and complex coacervatian, interfacial polymerization, and other methods well known to those of ordinary skill in the art. Particles may be made using methods for making microspheres or microcapsules known in the art.

Dry powder salt formulations that contain a calcium salt generally contain at least about 5% calcium salt by weight, 10% calcium salt by weight, about 15% calcium salt by weight, at least about 19.5% calcium salt by weight, at least about 20% calcium salt by weight, at least about 22% calcium salt by weight, at least about 25.5% calcium salt by weight, at least about 30% calcium salt by weight, at least about 37% calcium salt by weight, at least about 40% calcium salt by weight, at least about 48.4% calcium salt by weight, at least about 50% calcium salt by weight, at least about 60% calcium salt by weight, at least about 70% calcium salt by weight, at least about 75% calcium salt by weight, at least about 80% calcium salt by weight, at least about 85% calcium salt by weight, at least about 90% calcium salt by weight, or at least about 95% calcium salt by weight.

Alternatively or in addition, such dry powder formulations may contain a calcium salt which provides Ca⁺² in an amount of at least about 5% Ca⁺² by weight, at least about 7% Ca⁺² by weight, at least about 10% Ca⁺² by weight, at least about 11% Ca⁺² by weight, at least about 12% Ca⁺² by weight, at least about 13% Ca⁺² by weight, at least about 14% Ca⁺² by weight, at least about 15% Ca⁺² by weight, at least about 17% Ca⁺² by weight, at least about 20% Ca⁺² by weight, at least about 25% Ca⁺² by weight, at least about 30% Ca⁺² by weight, at least about 35% Ca⁺² by weight, at least about 40% Ca⁺² by weight, at least about 45% Ca⁺² by weight, at least about 50% Ca⁺² by weight, at least about 55% Ca⁺² by weight, at least about 60% Ca⁺² by weight, at least about 65% Ca⁺² by weight or at least about 70% Ca⁺² by weight.

When a dry powder salt formulation contains a calcium salt and a sodium salt the amount of sodium salt in the dry powder formulation can be dependent upon the desired calcium:sodium ratio. For example, the dry powder formulation may contain at least about 1.6% sodium salt by weight, at least about 5% sodium salt by weight, at least about 10% sodium salt by weight, at least about 13% sodium salt by weight, at least about 15% sodium salt by weight, at least about 20% sodium salt by weight, at least about 24.4% sodium salt by weight, at least about 28% sodium salt by weight, at least about 30% sodium salt by weight, at least about 30.5% sodium salt by weight, at least about 35% sodium salt by weight, at least about 40% sodium salt by weight, at least about 45% sodium salt by weight, at least about 50% sodium salt by weight, at least about 55% sodium salt by weight, or at least about 60% sodium salt by weight.

Alternatively or in addition, dry powder salt formulations may contain a sodium salt which provides Na⁺ in an amount of at least about 0.1% Na⁺ by weight, at least about 0.5% Na⁺ by weight, at least about 1% Na⁺ by weight, at least about 2% Na⁺ by weight, at least about 3% Na⁺ by weight, at least about 4% Na⁺ by weight, at least about 5% Na⁺ by weight, at least about 6% Na⁺ by weight, at least about 7% Na⁺ by weight, at least about 8% Na⁺ by weight, at least about 9% Na⁺ by weight, at least about 10% Na⁺ by weight, at least about 11% Na⁺ by weight, at least about 12% Na⁺ by weight, at least about 14% Na⁺ by weight, at least about 16% Na⁺ by weight, at least about 18% Na⁺ by weight, at least about 20% Na⁺ by weight, at least about 22% Na⁺ by weight, at least about 25% Na⁺ by weight, at least about 27% Na⁺ by weight, at least about 29% Na⁺ by weight, at least about 32% Na⁺ by weight, at least about 35% Na⁺ by weight, at least about 40% Na⁺ by weight, at least about 45% Na⁺ by weight, at least about 50% Na⁺ by weight, or at least about 55% Na⁺ by weight.

Preferred excipients for dry powder salt formulations (such as the hydrophobic amino acid leucine) can be present in the formulations in an amount of about 50% or less (w/w). For example, a dry powder formulation may contain the amino acid leucine in an amount of about 50% or less by weight, about 45% or less by weight, about 40% or less by weight, about 35% or less by weight, about 30% or less by weight, about 25% or less by weight, about 20% or less by weight, about 18% or less by weight, about 16% or less by weight, about 15% or less by weight, about 14% or less by weight, about 13% or less by weight, about 12% or less by weight, about 11% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, or about 1% or less by weight. Exemplary excipients may include leucine, maltodextrin, mannitol, any combination of leucine, maltodextrin, and mannitol, or any other excipients disclosed herein or commonly used in the art.

The compositions of some preferred salt compositions are presented in Table 1. The compositions disclosed in Table 1 are non-limiting examples of salt compositions that can be administered in accordance with the methods of the invention.

TABLE 1
Liquid formulations of Calcium Chloride

| Formu- | Tonicity | | | | |
|---|---|---|---|---|---|
| lation | (1X = | CaCl₂ | CaCl₂ | NaCl | NaCl |
| # | isotonic) | (% w/v) | (M) | (% w/v) | (M) |
| 1 | 2X | 1.3 | 0.12 | 0.90 | 0.15 |
| 2 | 4X | 4.2 | 0.38 | 0.90 | 0.15 |
| 3 | 6X | 6.4 | 0.58 | 0.90 | 0.15 |
| 4 | 8X | 9.0 | 0.81 | 0.90 | 0.15 |
| 5 | 11X | 13 | 1.2 | 0.90 | 0.15 |
| 6 | 2X | 2.6 | 0.23 | n.a. | n.a. |
| 7 | 5X | 6.4 | 0.58 | n.a. | n.a. |
| 8 | 10X | 13 | 1.2 | n.a. | n.a. |
| 9 | 0.5X | 0.59 | 0.053 | 0.040 | 0.0070 |
| 10 | 1X | 1.2 | 0.11 | 0.080 | 0.013 |
| 11 | 2X | 2.4 | 0.21 | 0.16 | 0.027 |
| 12 | 4X | 4.7 | 0.42 | 0.31 | 0.053 |
| 13 | 8X | 9.4 | 0.85 | 0.62 | 0.11 |
| 14 | 0.5X | 0.32 | 0.029 | 0.23 | 0.039 |
| 15 | 1X | 0.65 | 0.058 | 0.45 | 0.077 |
| 16 | 4X | 2.6 | 0.23 | 1.8 | 0.31 |
| 17 | 8X | 5.2 | 0.47 | 3.6 | 0.62 |

Liquid formulations of Calcium Lactate

| | Tonicity | Ca- | Ca- | | |
|---|---|---|---|---|---|
| Formu- | (1X = | lactate | lactate | NaCl | NaCl |
| lation | isotonic) | (%) | (M) | (%) | (M) |
| 18 | 0.5X | 0.76 | 0.035 | 0.23 | 0.039 |
| 19 | 1X | 1.5 | 0.070 | 0.45 | 0.077 |
| 20 | 2X | 3.0 | 0.14 | 0.90 | 0.15 |
| 21 | 4X | 6.1 | 0.28 | 1.8 | 0.31 |
| 22 | 6X | 9.1 | 0.42 | 2.7 | 0.46 |
| 23 | 8X | 12 | 0.56 | 3.6 | 0.62 |
| 24 | 0.5X | 1.4 | 0.065 | 0.048 | 0.0082 |
| 25 | 1X | 2.9 | 0.13 | 0.10 | 0.016 |
| 26 | 2X | 5.7 | 0.26 | 0.19 | 0.033 |
| 27 | 3X | 9.0 | 0.41 | 0.30 | 0.052 |
| 28 | 4X | 11 | 0.52 | 0.38 | 0.065 |
| 29 | 8X | 23 | 1.0 | 0.77 | 0.13 |

Powder formulations

| | Formulation composition | | | | | |
|---|---|---|---|---|---|---|
| Formu- | | Excip- | | Calcium | | Sodium |
| lation | Excip- | ient | Calcium | salt | Sodium | salt |
| # | ient | (wt %) | salt | (wt %) | salt | (wt %) |
| 30 | Leucine | 50.0 | Calcium | 29.5 | Sodium | 20.5 |
| | | | chloride | | chloride | |
| 31 | Leucine | 50.0 | Calcium | 33.8 | Sodium | 16.2 |
| | | | acetate | | chloride | |
| 32 | Leucine | 50.0 | Calcium | 37.0 | Sodium | 13.0 |
| | | | lactate | | chloride | |
| 33 | Leucine | 50.0 | Calcium | 22.0 | Sodium | 28.0 |
| | | | chloride | | sulfate | |
| 34 | Leucine | 50.0 | Calcium | 19.5 | Sodium | 30.5 |
| | | | chloride | | citrate | |
| 35 | Leucine | 10.0 | Calcium | 66.6 | Sodium | 23.4 |
| | | | lactate | | chloride | |
| 36 | Leucine | 10.0 | Calcium | 39.6 | Sodium | 50.4 |
| | | | chloride | | sulfate | |
| 37 | Leucine | 10.0 | Calcium | 35.1 | Sodium | 54.9 |
| | | | chloride | | citrate | |
| 38 | n.a. | n.a. | Calcium | 74.0 | Sodium | 26.0 |
| | | | lactate | | chloride | |
| 39 | n.a. | n.a. | Calcium | 44.0 | Sodium | 56.0 |
| | | | chloride | | sulfate | |
| 40 | n.a. | n.a. | Calcium | 39.0 | Sodium | 61.0 |
| | | | chloride | | citrate | |
| 41 | Leucine | 10.0 | Calcium | 58.6 | Sodium | 31.4 |
| | | | lactate | | chloride | |
| 42 | Maltodextrin | 10.0 | Calcium | 58.6 | Sodium | 31.4 |
| | | | lactate | | chloride | |
| 43 | Mannitol | 10.0 | Calcium | 58.6 | Sodium | 31.4 |
| | | | lactate | | chloride | |
| 44 | Lactose | 10.0 | Calcium | 58.6 | Sodium | 31.4 |
| | | | lactate | | chloride | |
| 45 | Half leucine | 10.0 | Calcium | 58.6 | Sodium | 31.4 |
| | and half | | lactate | | chloride | |
| | maltodextrin | | | | | |
| | (wt basis) | | | | | |
| 46 | Half leucine | 20.0 | Calcium | 52.1 | Sodium | 27.9 |
| | and half | | lactate | | chloride | |
| | maltodextrin | | | | | |
| | (wt basis) | | | | | |
| 47 | Leucine | 20.0 | Calcium | 52.1 | Sodium | 27.9 |
| | | | lactate | | chloride | |
| 48 | Leucine | 12.0 | Calcium | 57.3 | Sodium | 30.7 |
| | | | lactate | | chloride | |
| 49 | Leucine | 8.0 | Calcium | 59.9 | Sodium | 32.1 |
| | | | lactate | | chloride | |

n.a. not applicable

### Methods

Treatment of Pneumonia

The invention provides methods for the treatment, prophylaxis and reduction in contagion of pneumonia (e.g., bacterial pneumonia, viral pneumonia). An effective amount of a salt formulation (i.e., one or more salts) is administered to an individual (e.g., a mammal, such as a human or other primate, or domesticated animal, such as pigs, cows, sheep, chickens) to treat, prevent or reduce contagion of pneumonia. Preferably, the salt formulation is administered by inhalation of an aerosol. The invention also provides methods for the treatment, prophylaxis and reduction in contagion of pneumonia in an individual with a chronic underlying respiratory disease (e.g., asthma, chronic bronchitis, chronic obstructive pulmonary disease, cystic fibrosis).

In one aspect, the invention is a method for treating pneumonia comprising administering to an individual that has pneumonia an effective amount of a salt formulation. The salt formulation is administered to the respiratory tract (e.g., lungs) of the individual. The individual may have pneumonia caused by a bacterial infection, such as an infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof. In particular embodiments, the individual is infected by Streptococcus pneumoniae, Klebsiella pneumoniae or Pseudomonas aeruginosa. In a more particular embodiment, the individual is infected by Streptococcus pneumoniae. The individual may have pneumonia caused by a viral infection, such as an infection by a virus selected from the group consisting of influenza virus, respiratory syncytial virus, adenovirus, metapneumovirus, cytomegalovirus and herpes simplex virus.

The individual may have community acquired pneumonia, such as pneumonia caused by infection by Streptococcus pneumoniae. The individual may have healthcare associated pneumonia, such as ventilator associated pneumonia.

Preferably, the method of treating pneumonia comprises administering to an individual that has pneumonia an effective amount of a calcium salt formulation. More preferably, the calcium salt formulation also comprises a sodium salt, such as sodium chloride. Suitable calcium salt formulations, including formulations that contain a calcium salt and a sodium salt, are described herein.

In particular embodiments, the invention is a method for treating VAP, comprising administering to the respiratory tract of a patient with VAP an effective amount of a calcium formulation as described herein. The calcium formulation is preferably administered to the respiratory tract of the individual as an aerosol, for example, using a nebulizer. For example, the calcium salt formulation can be administered to a patient on a mechanical ventilatory using a nebulizer that is connected to the inspiratory limb of the ventilator circuit. Preferably, the calcium salt formulation is administered to the patient with VAP at the time VAP is suspected, e.g., when purulent sputum is detected. If desired, the method can further comprise administering one or more antibiotics to the patient with VAP. Optionally, synergistic amounts of the salt formulation and the antibiotic are administered. Salt formulations such as calcium salt formulations and antibiotics can be synergistic when administered as co-therapeutic agents and can provide superior therapy, that results in less antibiotic being administered, better pathogen clearance, shortening the duration of antibiotic therapy or by decreasing the likelihood of emerging resistance. The antibiotics can be administered using any suitable mode of administration, such as orally, intravenously, or by inhalation. A clinician of ordinary skill will be able to determine whether the patient with VAP presents risk factors for multi-drug resistant (MDR) pathogens. When the patient does not present risk factors for MDR, in certain embodiments, the patient is administered one or more antibiotics selected from the group consisting of ceftriaxone, ampicillin-sulbactam, piperacillin-tazobactam, levofloxacin, moxifloxacin and ertapenem. When the patient presents risk factors for MDR pathogens, in certain embodiments, the patient is administered a combination of antibiotics, containing at least one antibiotic selected from cefepime, ceftazidime, imipenem, meropenem, doripenem, piperacillin-tazobactam, and aztreonam; at least one antibiotic selected from ciprofloxacin, levofloxacin, gentamicin, tobramycin and amikacin; and at least one antibiotic selected from linezolid and vancomycin.

In particular embodiments, the method comprises administering to the respiratory tract of a patient with VAP an effective amount of a calcium salt formulation and one or more antibiotics (such as tobramycin). The calcium salt formulation and the antibiotic can be administered as separate formulations, or can be components of a single formulation as described herein.

Prophylaxis of Pneumonia

In another aspect, the invention is a method for prophylaxis or prevention of pneumonia comprising administering to an individual at risk for pneumonia or at risk for infection by a pathogen (e.g., bacteria, virus) that causes pneumonia an effective amount of a salt formulation. The salt formulation is administered to the respiratory tract (e.g., lungs, respiratory airways) of the individual. The method can be used to prevent or to decrease the rate or incidence of infection by a pathogen (e.g., bacteria, virus) that causes pneumonia.

The individual to be treated may be at risk for infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof. In particular embodiments, the individual is at risk for infection by Streptococcus pneumoniae, Klebsiella pneumoniae or Pseudomonas aeruginosa. In a more particular embodiment, the individual is at risk for infection by Streptococcus pneumoniae. The individual may be at risk for infection by a virus selected from the group consisting of influenza virus, respiratory syncytial virus, adenovirus, metapneumovirus, cytomegalovirus and herpes simplex virus.

Generally, individuals are at risk for infection by a pathogen (e.g., virus, bacteria) that causes infection of the respiratory tract when they are exposed to such a pathogen more frequently then the general population, or have a diminished capacity to resist infection. Individuals who are at risk for such an infection include, for example, health care workers, individuals who are immunosuppressed (e.g., medically, due to other infections, or for other reasons), patients in an intensive care unit, elderly and young (e.g., infants) individuals, individuals with chronic underlying respiratory disease (e.g., asthma, chronic bronchitis, chronic obstructive pulmonary disease, cystic fibrosis) individuals who have had surgery or traumatic injury, and care givers and family members of infected persons.

Accordingly, the method is suitable for prophylaxis or prevention of CAP, such as pneumonia caused by infection by Streptococcus pneumoniae. The method is particularly suitable for prophylaxis or prevention of healthcare associated pneumonia, such as ventilator associated pneumonia. For example, healthcare workers can be administered a salt formulation as described herein to reduce or prevent the rate of infection by a pathogen that causes pneumonia. In particular embodiments of this aspect, the invention is a method for the prophylaxis or prevention of ventilator associated pneumonia, comprising administering to an individual who is being ventilated an effective amount of a salt formulation. The salt formulation is administered to the respiratory tract (e.g., lungs) of the individual. The salt formulation can be administered prior to ventilation, during the course of ventilation (e.g., periodically while the individual is ventilated) and/or after ventilation is discontinued.

Preferably, the method of prophylaxis or prevention of pneumonia comprises administering an effective amount of a calcium salt formulation. More preferably, the calcium salt formulation also comprises a sodium salt, such as sodium chloride. Suitable calcium salt formulations, including formulations that contain a calcium salt and a sodium salt, are described herein.

In particular embodiments, the invention is a method for preventing or reducing the incidence of VAP, comprising administering to the respiratory tract of a patient at risk for developing VAP an effective amount a calcium formulation as described herein. Patients who are on mechanical ventilators are at risk for developing VAP, particularly those patients who will be mechanically ventilated for 48 hours or longer. The calcium formulation is preferably administered to the respiratory tract of the individual as an aerosol, for example, using a nebulizer. Preferably, the calcium salt formulation is administered to the patient at the time mechanical ventilation commences, e.g., at the time of intubation, and then periodically (e.g, once, twice, three or four times each day) while the patient remains on the mechanical ventilator. For example, the calcium salt formulation can be administered to a patient on a mechanical ventilator using a nebulizer that is connected to the inspiratory limb of the ventilator circuit. If desired, the method can further comprise administering one or more other therapeutic agents to prevent VAP to the patient, such as one or more antibiotics. The antibiotics can be administered using any suitable mode of administration, such as orally, intravenously, or by inhalation.

In particular embodiments, the method comprises administering to the respiratory tract of a patient at risk for developing VAP an effective amount of a calcium salt formulation and one or more antibiotics. The calcium salt formulation and the antibiotic can be administered as separate formulations, or can be components of a single formulation as described herein. Optionally, synergistic amounts of the salt formulation and the antibiotic are administered. Salt formulations such as calcium salt formulations and antibiotics can be synergistic when administered as co-therapeutic agents and can provide superior therapy, that results in less antibiotic being administered, better pathogen clearance, shortening the duration of antibiotic therapy or by decreasing the likelihood of emerging resistance.

Treatment of VAT

The invention provides methods for the treatment, prophylaxis and reduction in contagion of VAT. An effective amount of a salt formulation (i.e., one or more salts) is administered to an individual (e.g., a mammal, such as a human or other primate, or domesticated animal, such as pigs, cows, sheep, chickens) to treat, prevent or reduce contagion of VAT. Preferably, the salt formulation is administered by inhalation of an aerosol.

In one aspect, the invention is a method for treating VAT comprising administering to an individual that has VAT an effective amount of a salt formulation. The salt formulation is administered to the respiratory tract (e.g., lungs) of the individual. The individual may have VAT caused by a bacterial infection, such as an infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof. In particular embodiments, the individual is infected by Streptococcus pneumoniae, Klebsiella pneumoniae or Pseudomonas aeruginosa. In a more particular embodiment, the individual is infected by Streptococcus pneumoniae.

Preferably, the method of treating VAT comprises administering to an individual that has VAT an effective amount of a calcium salt formulation. More preferably, the calcium salt formulation also comprises a sodium salt, such as sodium chloride. Suitable calcium salt formulations, including formulations that contain a calcium salt and a sodium salt, are described herein. The calcium formulation is preferably administered to the respiratory tract of the individual as an aerosol, for example, using a nebulizer. For example, the calcium salt formulation can be administered to a patient on a mechanical ventilatory using a nebulizer that is connected to the inspiratory limb of the ventilator circuit. Preferably, the calcium salt formulation is administered to the patient with VAT at the time VAT is suspected, e.g., when clinical signs of lower respiratory tract infection appear, such as when purulent sputum is detected. If desired, the method can further comprise administering one or more antibiotics to the patient with VAT. The antibiotics can be administered using any suitable mode of administration, such as orally, intravenously, or by inhalation. Optionally, synergistic amounts of the salt formulation and the antibiotic are administered. Salt formulations such as calcium salt formulations and antibiotics can be synergistic when administered as co-therapeutic agents and can provide superior therapy, that results in less antibiotic being administered, better pathogen clearance, shortening the duration of antibiotic therapy or by decreasing the likelihood of emerging resistance.

The calcium salt formulation may be administered to a patient to reduce contagion of VAT (e.g., to protect healthcare workers prior to weaning the patient off of a mechanical ventilatory). The calcium salt formulation may be administered to prevent spread of pathogens through physical contact between an intubated patient and health-care workers and transmission of pathogens via mucus and other bodily secretions

A clinician of ordinary skill will be able to determine whether the patient with VAT presents risk factors for multi-drug resistant (MDR) pathogens. When the patient does not present risk factors for MDR, in certain embodiments, the patient is administered one or more antibiotics selected from the group consisting of ceftriaxone, ampicillin-sulbactam, piperacillin-tazobactam, levofloxacin, moxifloxacin and ertapenem. When the patient presents risk factors for MDR pathogens, in certain embodiments, the patient is administered a combination of antibiotics, containing at least one antibiotic selected from cefepime, ceftazidime, imipenem, meropenem, doripenem, piperacillin-tazobactam, and aztreonam; at least one antibiotic selected from ciprofloxacin, levofloxacin, gentamicin, tobramycin and amikacin; and at least one antibiotic selected from linezolid and vancomycin.

In particular embodiments, the method compres administering to the respiratory tract of a patient with VAT an effective amount of a calcium salt formulation and one or more antibiotics. The calcium salt formulation and the antibiotic can be administered as separate formulations, or can be components of a single formulation as described herein.

Prophylaxis of VAT

In another aspect, the invention is a method for prophylaxis or prevention of VAT comprising administering to an individual at risk for VAT or at risk for infection by a pathogen (e.g., bacteria, virus) that causes VAT, such as an intubated patient, an effective amount of a salt formulation. The salt formulation is administered to the respiratory tract (e.g., lungs, respiratory airways) of the individual. The method can be used to prevent or to decrease the rate or incidence of VAT or infection by a pathogen (e.g., bacteria, virus) that causes VAT.

The individual to be treated may be at risk for VAT associated with infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp., Mycobacterium and combinations thereof. In particular embodiments, the individual is at risk for VAT associated with infection by Streptococcus pneumoniae, Klebsiella pneumoniae or Pseudomonas aeruginosa. In a more particular embodiment, the individual is at risk for VAT associated with infection by Streptococcus pneumoniae.

In particular embodiments of this aspect, the invention is a method for the prophylaxis or prevention of VAT, comprising administering to an individual who is being ventilated an effective amount of a salt formulation. The salt formulation is administered to the respiratory tract (e.g., lungs) of the individual. The salt formulation can be administered prior to ventilation, during the course of ventilation (e.g., periodically while the individual is ventilated) and/or after ventilation is discontinued.

Preferably, the method of prophylaxis or prevention of VAT comprises administering an effective amount of a calcium salt formulation. More preferably, the calcium salt formulation also comprises a sodium salt, such as sodium chloride. Suitable calcium salt formulations, including formulations that contain a calcium salt and a sodium salt, are described herein.

In particular embodiments, the invention is a method for preventing or reducing the incidence of VAT, comprising administering to the respiratory tract of a patient at risk for developing VAT an effective amount a calcium formulation as described herein. Patients who are on mechanical ventilators are at risk for developing VAT, particularly those patients who will be mechanically ventilated for 48 hours or longer. The calcium formulation is preferably administered to the respiratory tract of the individual as an aerosol, for example, using a nebulizer. Preferably, the calcium salt formulation is administered to the patient at the time mechanical ventilation commences, e.g., at the time of intubation, and then periodically (e.g, once, twice, three or four times each day) while the patient remains on the mechanical ventilator. For example, the calcium salt formulation can be administered to a patient on a mechanical ventilator using a nebulizer that is connected to the inspiratory limb of the ventilator circuit. If desired, the method can further comprise administering one or more other therapeutic agents to prevent VAT to the patient, such as one or more antibiotics. The antibiotics can be administered using any suitable mode of administration, such as orally, intravenously, or by inhalation.

In particular embodiments, the method comprises administering to the respiratory tract of a patient at risk for developing VAT an effective amount of a calcium salt formulation and one or more antibiotics. The calcium salt formulation and the antibiotic can be administered as separate formulations, or can be components of a single formulation as described herein.

### Treatment of Bacterial Infections of the Respiratory Tract

The invention provides methods for the treatment (including prophylactic treatment) and reduction in contagion of a bacterial infection of the respiratory tract (e.g., pneumonia). An effective amount of a salt formulation (i.e., one or more salts) and an antibiotic agent is administered to an individual (e.g., a mammal, such as a human or other primate, or domesticated animal, such as pigs, cows, sheep, chickens) to treat, prevent or reduce contagion of a bacterial infection of the respiratory tract (e.g., pneumonia). Preferably, the salt formulation is administered by inhalation of an aerosol.

In one aspect, the invention is a method for treating a bacterial infection of the respiratory tract (e.g., pneumonia) comprising administering to an individual that has a bacterial infection of the respiratory tract an effective amount of a salt formulation and an antibiotic. The salt formulation is preferably administered to the respiratory tract (e.g., lungs) of the individual. The antibiotic can be administered by any suitable route, such as orally, systemically or by inhalation. Optionally, synergistic amounts of the salt formulation and the antibiotic are administered. Salt formulations such as calcium salt formulations and antibiotics can be synergistic when administered as co-therapeutic agents and can provide superior therapy, that results in less antibiotic being administered, better pathogen clearance, shortening the duration of antibiotic therapy or by decreasing the likelihood of emerging resistance. For example, the individual's respiratory tract may be infected with a pathogen selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp., Mycobacterium and combinations thereof. In a more particular embodiment, the individual is infected by Streptococcus pneumoniae. The individual may have a viral infection, such as an infection by a virus selected from the group consisting of influenza virus, respiratory syncytial virus, adenovirus, metapneumovirus, cytomegalovirus and herpes simplex virus.

In certain embodiments, ventilator associate pneumonia (VAP), ventilator associated tracheobronchitis (VAT), or hospital acquired pneumonia (HAP), is caused by pneumoniae, S. pneumoniae, S. aureus, non-typeable Haemophilus influenzae (NTHI), psuedominas aeruginosa, Acinetobacter spp., E coli, Candida spp (a fungus), Serratia, Enterobacter spp, and Stenotrophomonas. Alternatively, VAP or VAT can be caused by Gram-positive or Gram-negative bacteria associated with causing pneumonia.

In certain embodiments, community associated pneumonia (CAP) is caused by at least one of the following bacteria: Moraxella catarralis, Mycoplasma pneumoniae, Chlamydophilia pneumonia, or Chlamydia pneumoniae, strep pneumonia, Haemophilus influenzae, chlamydophia, mycoplasma, and Legionella. Alternatively, or in addition to the previously mentioned bacteria, CAP may also be cause by at least one of the following fungi: Coccidiomycosis, histoplasmosis, and cryptococcocus. Alternatively, CAP can be caused by Gram-positive or Gram-negative bacteria associated with causing pneumonia.

Preferably, the method of treating pneumonia comprises administering to an individual that has pneumonia an effective amount of a calcium salt formulation. More preferably, the calcium salt formulation also comprises a sodium salt, such as sodium chloride. Suitable calcium salt formulations, including formulations that contain a calcium salt and a sodium salt, are described herein. If desired, the formulation can further comprise one or more antibiotics.

In another aspect, the invention is a method for prophylaxis or prevention of a bacterial infection of the respiratory tract (e.g., pneumonia) comprising administering to an individual at risk for a bacterial infection of the respiratory tract (e.g., pneumonia) or at risk for infection by a pathogen that causes a bacterial infection of the respiratory tract (e.g., pneumonia) an effective amount of a salt formulation and an antibiotic. The salt formulation is administered to the respiratory tract (e.g., lungs, respiratory airways) of the individual. The antibiotic can be administered by any suitable route, such as orally, systemically or by inhalation. Optionally, synergistic amounts of the salt formulation and the antibiotic are administered. The method can be used to prevent or to decrease the rate or incidence of infection by a pathogen that causes a bacterial infection of the respiratory tract (e.g., pneumonia).

The individual to be treated may be at risk for infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp. and combinations thereof. In a more particular embodiment, the individual is at risk for infection by Streptococcus pneumoniae. The individual may be at risk for infection by a virus selected from the group consisting of influenza virus, respiratory syncytial virus, adenovirus, metapneumovirus, cytomegalovirus and herpes simplex virus.

Accordingly, the method is suitable for prophylaxis or prevention of CAP, such as pneumonia caused by infection by Streptococcus pneumoniae. The method is particularly suitable for prophylaxis or prevention of healthcare associated pneumonia, such as ventilator associated pneumonia. For example, healthcare workers can be administered a salt formulation as described herein to reduce or prevent the rate of infection by a pathogen that causes pneumonia. In particular embodiments of this aspect, the invention is a method for the prophylaxis or prevention of ventilator associated pneumonia, comprising administering to an individual who is being ventilated an effective amount of a salt formulation. The salt formulation is administered to the respiratory tract (e.g., lungs) of the individual. The salt formulation can be administered prior to ventilation, during the course of ventilation (e.g., periodically while the individual is ventilated) and/or after ventilation is discontinued.

Preferably, the method of prophylaxis or prevention of pneumonia comprises administering an effective amount of a calcium salt formulation. More preferably, the calcium salt formulation also comprises a sodium salt, such as sodium chloride. Suitable calcium salt formulations, including formulations that contain a calcium salt and a sodium salt, are described herein.

Reducing Contagion

The invention provides methods for reducing contagion (e.g., reducing transmission) of pneumonia (e.g., bacterial pneumonia, viral pneumonia), such as VAP, or VAT comprising administering to an individual infected with a pathogen that causes pneumonia or VAT or at risk for pneumonia or VAT, or at risk for infection by a pathogen (e.g., bacteria, virus) that causes pneumonia or VAT, an effective amount of a salt formulation. The salt formulation is administered to the respiratory tract (e.g., lungs) of the individual. In particular embodiments, the salt formulation is administered to reduce contagion that occurs when a patient is being weaned from mechanical ventilation. In these situations, the respirator is disconnected but the intubation tube remains in place. The intubation tube has a narrower diameter than the airway and the velocity of air passing through the tube is high, creating a risk that infection particles will be exhaled.

The individual may have pneumonia or VAT caused by or associated with a bacterial infection or be at risk for such an infection as described herein. For example, the individual may be infected by or at risk for infection by a bacteria selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Stenotrophomonas maltophilia, Burkholderia spp., Mycobacterium and combinations thereof. In particular embodiments, the individual is infected by or at risk of infection by Streptococcus pneumoniae, Klebsiella pneumoniae or Pseudomonas aeruginosa. In a more particular embodiment, the individual is infected by or at risk for infection by Streptococcus pneumoniae. The individual may be infected by or at risk for infection by a virus selected from the group consisting of influenza virus, respiratory syncytial virus, adenovirus, metapneumovirus, cytomegalovirus and herpes simplex virus.

The individual may have or be at risk for acquiring community acquired pneumonia, such as pneumonia caused by infection by Streptococcus pneumoniae. The individual may have or be at risk for acquiring healthcare associated pneumonia, such as ventilator associated pneumonia, or the individual may be at risk for acquiring VAT.

Preferably, the method for reducing contagion of pneumonia or VAT comprises administering to an individual an effective amount of a calcium salt formulation. More preferably, the calcium salt formulation also comprises a sodium salt, such as sodium chloride. Suitable calcium salt formulations, including formulations that contain a calcium salt and a sodium salt, are described herein.

### Administering Salt Formulations

The salt formulations are intended for administration to the respiratory tract (e.g., to the mucosal surface of the respiratory tract), and can be administered in any suitable form, such as a solution, a suspension, a spray, a mist, a foam, a gel, a vapor, droplets, particles, or a dry powder form. Preferably the salt formulation is aerosolized for administration to the respiratory tract. Salt formulations can be aerosolized for administration via the oral airways using any suitable method and/or device, and many suitable methods and devices are conventional and well-known in the art. For example, salt formulations can be aerosolized using a metered dose inhaler (e.g., a pressurized metered dose inhaler (pMDI) including HFA propellant, or a non-HFA propellant) with or without a spacer or holding chamber, a nebulizer, an atomizer, a continuous sprayer, an oral spray or a dry powder inhaler (DPI). Salt formulations can be aerosolized for administration via the nasal airways using a nasal pump or sprayer, a metered dose inhaler (e.g., a pressurized metered dose inhaler (pMDI) including HFA propellant, or a non-HFA propellant) with or without a spacer or holding chamber, a nebulizer with or without a nasal adapter or prongs, an atomizer, a continuous sprayer, or a DPI. Salt formulations can also be delivered to the nasal mucosal surface via, for example, nasal wash and to the oral mucosal surfaces via, for example, an oral wash. Salt formulations can be delivered to the mucosal surfaces of the sinuses via, for example, nebulizers with nasal adapters and nasal nebulizers with oscillating or pulsatile airflows.

The geometry of the airways is an important consideration when selecting a suitable method for producing and delivering aerosols of salt formulations to the lungs. The lungs are designed to entrap particles of foreign matter that are breathed in, such as dust. There are three basic mechanisms of deposition: impaction, sedimentation, and Brownian motion (J. M. Padfield. 1987. In: D. Ganderton & T. Jones eds. Drug Delivery to the Respiratory Tract, Ellis Harwood, Chicherster, U.K.). Impaction in the upper airways occurs when particles are unable to stay within the air stream, particularly at airway branches. Impacted particles are adsorbed onto the mucus layer covering bronchial walls and eventually cleared from the lungs by mucocilliary action. Impaction mostly occurs with particles over 5 μm in aerodynamic diameter. Smaller particles (those less than about 3 μm in aerodynamic diameter) tend to stay within the air stream and to be advected deep into the lungs. Sedimentation often occurs in the lower respiratory system where airflow is slower. Very small particles (those less than about 0.6 μm) can deposit by Brownian motion. Deposition by Brownian motion is generally undesirable because deposition cannot be targeted to the alveoli (N. Worakul & J. R. Robinson. 2002. In: Polymeric Biomaterials, 2^{nd} Ed. S. Dumitriu ed. Marcel Dekker. New York).

For administration, a suitable method (e.g., nebulization, dry powder inhaler) is selected to produce aerosols with the appropriate particle size for preferential delivery to the desired region of the respiratory tract, such as the deep lung (generally particles between about 0.6 microns and 5 microns in diameter), the upper airway (generally particles of about 3 microns or larger diameter), or the deep lung and the upper airway.

An effective amount of salt formulation is administered to an individual in need thereof, such as an individual who has pneumonia (bacterial pneumonia or viral pneumonia), such as VAP, pneumonia-like symptoms, VAT or who is at risk for infection by a pathogen that causes pneumonia or VAT. Individuals who are hospitalized, and particularly those who are ventilated, are at risk for infection by pathogens that cause pneumonia. An “effective amount” of salt formulation is administered. An effective amount is an amount that is sufficient to achieve the desired therapeutic or prophylactic effect, such as an amount sufficient to reduce pneumonia-like symptoms, to reduce pathogens in an individual, to inhibit pathogens passing through the lung mucus or airway lining fluid, to decrease the incidence or rate of infection with pathogens that cause pneumonia, to decrease the shedding of exhaled particles containing pathogens that cause pneumonia, and/or to increase mucociliary clearance (Groth et al, Thorax, 43(5):360-365 (1988)). Because the salt formulations are administered to the respiratory tract (e.g., lungs), generally by inhalation, the dose that is administered is related to the composition of the salt formulation (e.g., calcium salt concentration), the rate and effecience of aerosolization (e.g., nebulization rate and efficiency), and the time of exposure (e.g., nebulization time). For example, substantially equivalent doses can be administered using a concentrated liquid salt formulation and a short (e.g., 5 minutes) nebulization time, or using a dilute liquid salt formulation and a long (e.g., 30 minutes or more) nebulization time, or using a dry powder formulation and a dry powder inhaler. The clinician of ordinary skill can determine appropriate dosage based on these considerations and other factors, for example, the individual's age, sensitivity, tolerance and overall well-being. The salt formulations can be administered in a single dose or multiple doses as indicated.

As described herein, it is believed that the therapeutic and prophylactic effects of the salt formulations are the result of an increased amount of cation (the cation of the salt, such as Ca²⁺) in the respiratory tract (e.g., lung) following administration of a salt formulation. Accordingly, since the amount of cation provided can vary depending upon the particular salt selected, dosing can be based on the desired amount of cation to be delivered to the lung. For example, one mole of calcium chloride (CaCl₂) dissociates to provide one mole of ca²⁺, but one mole of tricalcium phosphate (Ca₃(PO₄)₂) can provide three moles of ca²⁺. Generally, an effective amount of a salt formulation will deliver a dose of about 0.001 mg Ca²⁺/kg body weight/dose to about 2 mg Ca⁺²/kg body weight/dose, about 0.002 mg Ca⁺²/kg body weight/dose to about 2 mg Ca⁺²/kg body weight/dose, about 0.005 mg Ca⁺²/kg body weight/dose to about 2 mg Ca⁺²/kg body weight/dose, about 0.01 mg Ca⁺²/kg body weight/dose to about 2 mg Ca⁺²/kg body weight/dose, about 0.01 mg Ca⁺²/kg body weight/dose to about 60 mg Ca⁺²/kg body weight/dose, about 0.01 mg Ca⁺²/kg body weight/dose to about 50 mg Ca⁺²/kg body weight/dose, about 0.01 mg Ca⁺²/kg body weight/dose to about 40 mg Ca⁺²/kg body weight/dose, about 0.01 mg Ca⁺²/kg body weight/dose to about 30 mg Ca⁺²/kg body weight/dose, about 0.01 mg Ca⁺²/kg body weight/dose to about 20 mg Ca⁺²/kg body weight/dose, about 0.01 mg Ca⁺²/kg body weight/dose to about 10 mg Ca⁺²/kg body weight/dose, about 0.01 mg Ca⁺²/kg body weight/dose to about 5 mg Ca⁺²/kg body weight/dose, about 0.01 mg Ca⁺²/kg body weight/dose to about 2 mg Ca⁺²/kg body weight/dose, about 0.02 mg Ca⁺²/kg body weight/dose to about 2 mg Ca⁺²/kg body weight/dose, about 0.03 mg Ca⁺²/kg body weight/dose to about 2 mg Ca⁺²/kg body weight/dose, about 0.04 mg Ca⁺²/kg body weight/dose to about 2 mg Ca⁺²/kg body weight/dose, about 0.05 mg Ca⁺²/kg body weight/dose to about 2 mg Ca⁺²/kg body weight/dose, about 0.1 mg Ca⁺²/kg body weight/dose to about 2 mg Ca⁺²/kg body weight/dose, about 0.1 mg Ca⁺²/kg body weight/dose to about 1 mg Ca⁺²/kg body weight/dose, about 0.1 mg Ca⁺²/kg body weight/dose to about 0.5 mg Ca⁺²/kg body weight/dose, about 0.2 mg Ca⁺²/kg body weight/dose to about 0.5 mg Ca⁺²/kg body weight/dose, about 0.18 mg Ca⁺²/kg body weight/dose, about 0.001 mg Ca⁺²/kg body weight/dose, about 0.005 mg Ca⁺²/kg body weight/dose, about 0.01 mg Ca⁺²/kg body weight/dose, about 0.02 mg Ca⁺²/kg body weight/dose, or about 0.5 mg Ca⁺²/kg body weight/dose. In some embodiments, a salt formulation that comprises a calcium salt (e.g., calcium chloride, calcium lactate, calcium citrate) is administered in an amount sufficient to deliver a dose of about 0.1 mg Ca²⁺/kg body weight/dose to about 2 mg Ca²⁺/kg body weight/dose, or about 0.1 mg Ca²⁺/kg body weight/dose to about 1 mg Ca²⁺/kg body weight/dose, or about 0.1 mg Ca²⁺/kg body weight/dose to about 0.5 mg Ca²⁺/kg body weight/dose, or about 0.18 mg Ca²⁺/kg body weight/dose.

In some embodiments the amount of calcium delivered to the respiratory tract (e.g., lungs, repiratory airway) is about 0.01 mg/kg body weight to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose.

In other embodiments the amount of calcium delivered to the upper respiratory tract (e.g., nasal cavity) is about 0.01 mg/kg body weight/dose to about 60 mg/kg body weight/dose, or about 0.01 mg/kg body weight/dose to about 50 mg/kg body weight/dose, about 0.01 mg/kg body weight/dose to about 40 mg/kg body weight/dose, about 0.01 mg/kg body weight/dose to about 30 mg/kg body weight/dose, about 0.01 mg/kg body weight/dose to about 20 mg/kg body weight/dose, 0.01 mg/kg body weight/dose to about 10 mg/kg body weight/dose, about 0.1 mg/kg body weight/dose to about 10 mg/kg body weight/dose, or about 1 mg/kg body weight/dose to about 10 mg/kg body weight/dose, or about 0.01 mg/kg body weight/dose to about 1 mg/kg body weight/dose, or about 0.1 mg/kg body weight/dose to about 1 mg/kg body weight/dose.

In some embodiments, a salt formulation that comprises a sodium salt (e.g., sodium chloride) is administered in an amount sufficient to deliver a dose of about 0.001 mg Na⁺/kg body weight/dose to about 10 mg Na⁺/kg body weight/dose, or about 0.01 mg Na⁺/kg body weight/dose to about 10 mg Na⁺/kg body weight/dose, or about 0.1 mg Na⁺/kg body weight/dose to about 10 mg Na⁺/kg body weight/dose, or about 1.0 mg Na⁺/kg body weight/dose to about 10 mg Na⁺/kg body weight/dose, or about 0.001 mg Na⁺/kg body weight/dose to about 1 mg Na⁺/kg body weight/dose, or about 0.01 mg Na⁺/kg body weight/dose to about 1 mg Na⁺/kg body weight/dose, about 0.1 mg Na⁺/kg body weight/dose to about 1 mg Na⁺/kg body weight/dose, about 0.2 mg Na⁺/kg body weight/dose to about 0.8 mg Na⁺/kg body weight/dose, about 0.3 mg Na⁺/kg body weight/dose to about 0.7 mg Na⁺/kg body weight/dose, or about 0.4 mg Na⁺/kg body weight/dose to about 0.6 mg Na⁺/kg body weight/dose.

In some embodiments the amount of sodium delivered to the respiratory tract (e.g., lungs, respiratory airway) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

In other embodiments the amount of sodium delivered to the upper respiratory tract (e.g., nasal cavity) is about 0.001 mg/kg body weight/dose to about 10 mg/kg body weight/dose, or about 0.01 mg/kg body weight/dose to about 10 mg/kg body weight/dose, or about 0.1 mg/kg body weight/dose to about 10 mg/kg body weight/dose, or about 1 mg/kg body weight/dose to about 10 mg/kg body weight/dose, or about 0.001 mg/kg body weight/dose to about 1 mg/kg body weight/dose, or about 0.01 mg/kg body weight/dose to about 1 mg/kg body weight/dose, or about 0.1 mg/kg body weight/dose to about 1 mg/kg body weight/dose.

Suitable intervals between doses that provide the desired therapeutic effect can be determined based on the severity of the condition (e.g., infection), overall well being of the subject and the subject's tolerance to the salt formulations and other considerations. Based on these and other considerations, a clinician can determine appropriate intervals between doses. Generally, a salt formulation is administered once, twice or three times a day, as needed.

If desired or indicated, a salt formulation can be administered with one or more other therapeutic agents, such as any one or more of the mucoactive agents, surfactants, cough suppressants, expectorants, steroids, brochodilators, antihistamines, antibiotics, antiviral agents described herein. The other therapeutic agents can be administered by any suitable route, such as orally, parenterally (e.g., intravenous, intraarterial, intramuscular, or subcutaneous injection), topically, by inhalation (e.g., intrabronchial, intranasal or oral inhalation, intranasal drops), rectally, vaginally, and the like. The salt formulation can be administered before, substantially concurrently with, or subsequent to administration of the other therapeutic agent. Preferably, the salt formulation and the other therapeutic agent are administered so as to provide substantial overlap of their pharmacologic activities.

As described herein, a pass through assay in which the migration of a pathogen through a mucus mimetic was used to model the process of respiratory tract infection (e.g., lung) infection in the studies described herein. The mucus mimetic used in the studies described herein is sodium alginate. Other suitable mucus mimetics that can be used in the pass through assay include locus bean gum crosslinked with sodium borate or other synthetic mimetics. Biologically derived mucus (e.g. mucus from a human or animal) can also be used in the pass through assay in place of the mucus mimetic.

The entire teachings of all documents cited herein are hereby incorporated herein by reference.

### EXEMPLIFICATION

### Example 1

### In vitro Studies

In vitro studies were conducted using a model of lung infection. A pass through assay in which the migration of a pathogen through a mucus mimetic was used. In this model, migration of pathogens across a mucus layer is assessed. The assay models the process of lung infection, because in order to establish infection and cause pneumonia in vivo pathogens must pass through the mucus layer lining the respiratory tract.

### Pass Through Assay

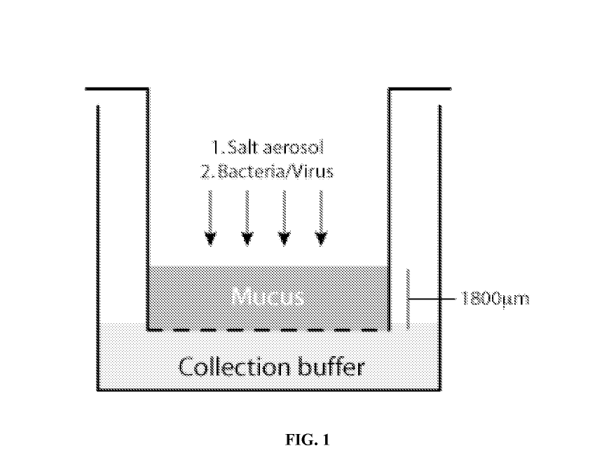

In this model, 200 μL of 4% sodium alginate (Sigma Aldrich, St. Louis, Mo.) was added to the apical surface of a 12 mm Transwell membrane (Costar, 3.0 μm pore size) and subsequently exposed to nebulized formulations. Liquid salt formulations were nebulized into the chamber using a sedimentation chamber, and allowed to settle by gravity over a 5 minute period. To control the concentration of salt formulation delivered to each set of wells, the number of nebulizations was varied. When multiple doses were delivered, salt formulations were nebulized at 5 minute intervals and aerosol was allowed to sediment in between each exposure. Following the delivery of salt formulations, 10 μL of Klebsiella pneumoniae, Streptococcus pneumoniae, Pseudomonas aeruginosa, Streptococcus aureus, or non-typeable Haemophilus influenzae (˜10⁷ CFU/mL in saline) was added to the apical surface of the mimetic. At various time points after the addition of bacteria, aliquots of the basolateral buffer were removed and the number of bacteria in each aliquot was determined by serially diluting and plating on blood agar plates. A schematic of this method is shown in FIG. 1. In some experiments, the concentration of salt that was delivered to each well was quantified. For this purpose, empty wells of the 12-well cell culture plate that were next to each Transwell and were exposed to the same dose of formulation were rinsed with sterile water. Samples were analyzed by osmometry and the concentration of calcium per unit area was determined from standard curves.

1A. Calcium Reduces the Movement of K. pneumoniae and S. pneumoniae Across Aodium Alginate Mucus Mimetic

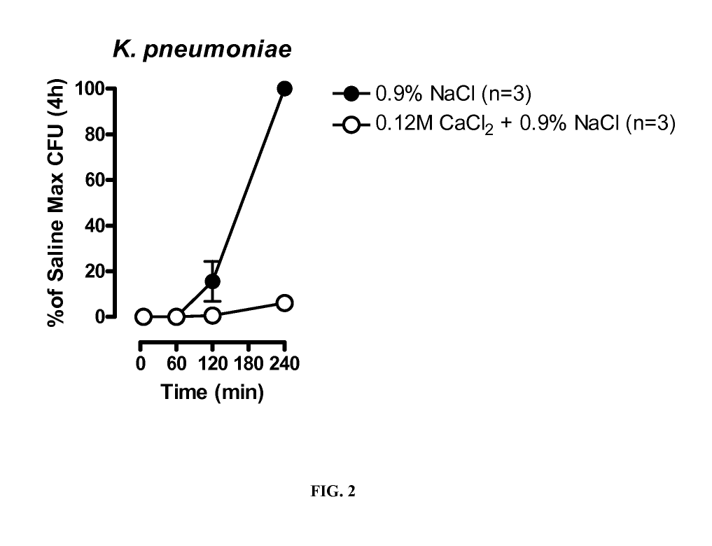

Sodium alginate mucus mimetic was exposed to aerosol generated from a 1.3% calcium chloride (0.12M) in 0.90% sodium chloride solution and the movement of K. pneumoniae from the apical to basolateral chamber across the mimetic was measured in three independent experiments. The approximate dose of calcium delivered to the mimetic in each experiment was 3-5 μg calcium per cm², based on historical measurements made with this formulation and the same experimental set up. In the saline treated control wells, bacteria were first recovered from the basolateral chamber 120 minutes after the addition of bacteria to the apical surface and the titer increased significantly between 120 and 240 minutes (FIG. 2). In contrast, the movement of bacteria through the calcium treated mimetic was delayed and significantly reduced [6%±2.4% of control at 4 h (n=3)]. When the area under the curve (AUC) for each test formulation was calculated for each experiment and compared statistically, there was a significant reduction in AUC for the calcium treatment compared to the saline control (p<0.001; Student t-test).

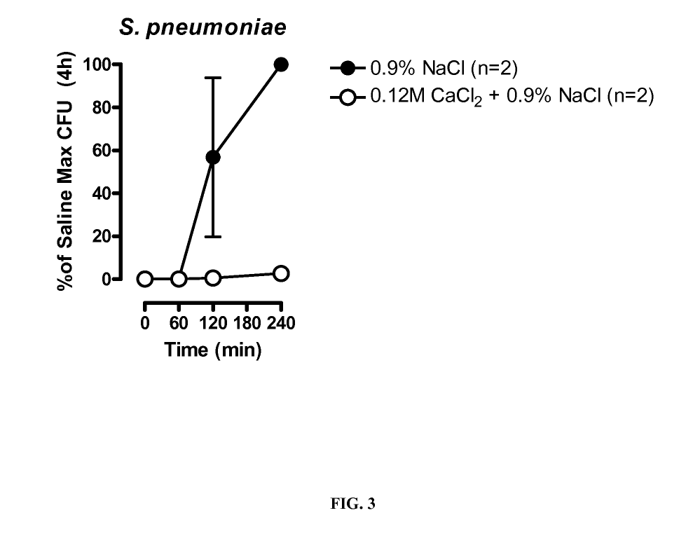

To determine if the findings made with K. pneumoniae (Gram-negative; rod shaped) would apply to a second bacterium of different shape, S. pneumoniae (Gram-positive; chains of diplococci) was tested in the same assay (FIG. 3). Similar to the results obtained with K. pneumoniae, exposure of the mimetic to calcium reduced the movement of S. pneumoniae [2.0%±2.0% of control at 4 h (n=3); p<0.05 for comparisons of AUC], indicating that the inhibition of bacterial movement by calcium treatment is applicable to multiple bacterial species. This effect is likely driven by changes in the biophysical properties of the sodium alginate mimetic caused by calcium, a notion supported by additional data generated using interfacial stress rheometry.

1B. Magnesium Reduces the Movement of K. pneumoniae Across Sodium Alginate Mucus Mimetic, but to a Lesser Extent than Calcium.

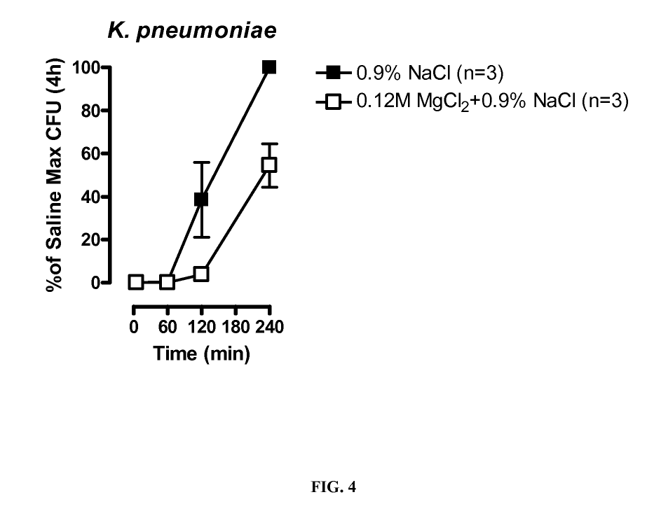

To determine if the effects observed with calcium treatment could be replicated with a second divalent cation, the effect of a nebulized magnesium chloride solution was evaluated. The formulation tested contained 0.12M magnesium chloride dissolved in 0.90% sodium chloride, which matched the molar concentration of magnesium chloride to that of calcium chloride. Like calcium chloride, the exposure of the sodium alginate mimetic to magnesium chloride aerosols reduced the movement of K. pneumoniae, however, the magnitude of the effect was significantly less than that observed for calcium chloride (p<0.05 for comparison of AUC for saline and magnesium treatment from three independent experiments; FIG. 4). Specifically, the titer of bacteria recovered from the basolateral chamber following magnesium chloride treatment was 54.5%±17.4% of the saline control at 240 minutes (n=3). A more substantial reduction by magnesium chloride was observed compared to the control at 120 minutes [(5.6%±2.4% of saline control at 120 minutes (n=3)]. The latter result suggests that magnesium chloride treatment may initially delay either the entry or movement of the bacteria in the mimetic, but that the effect is overcome more quickly than when calcium chloride was used.

1C. Zinc and Aluminum Reduce the Movement of K. pneumoniae Across Sodium Alginate Mucus Mimetic, but to a Lesser Extent than Calcium.

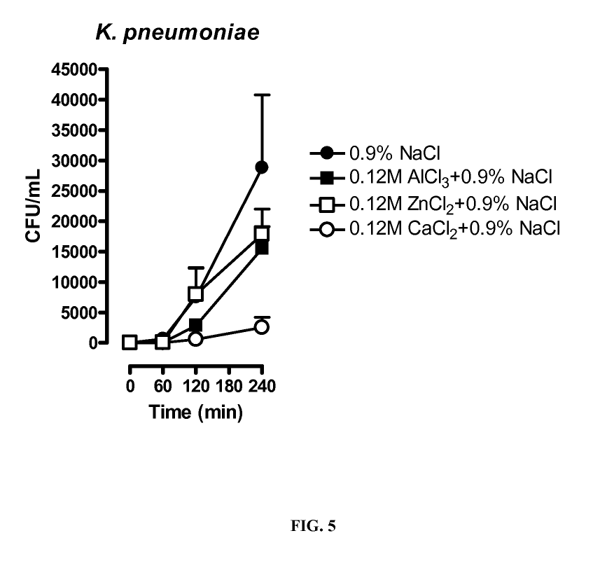

To further test if a relationship between valency and the inhibition of bacterial movement exists, additional tests were performed in which sodium alginate was exposed to either 0.12M zinc (divalent cation) chloride or 0.12M aluminum (trivalent cation) chloride solutions made in 0.90% sodium chloride. Formulations were delivered as above, except that wells were exposed in triplicate and a single experiment was performed. Similar to the magnesium chloride treatment, both zinc chloride and aluminum chloride had a modest effect on bacterial movement across the mimetic (˜50% of control at 4 hours), although the result was not statistically significant due to variability in one of the control wells. (FIG. 5)

The data presented above demonstrate that the movement of bacteria across a mucus layer can be impacted by changing the biophysical properties of the material.

1D. Prophylactic Exposure of Sodium Alginate Mimetic to Calcium Chloride Inhibits the Movement of K. pneumoniae Across Sodium Alginate Mucus Mimetic

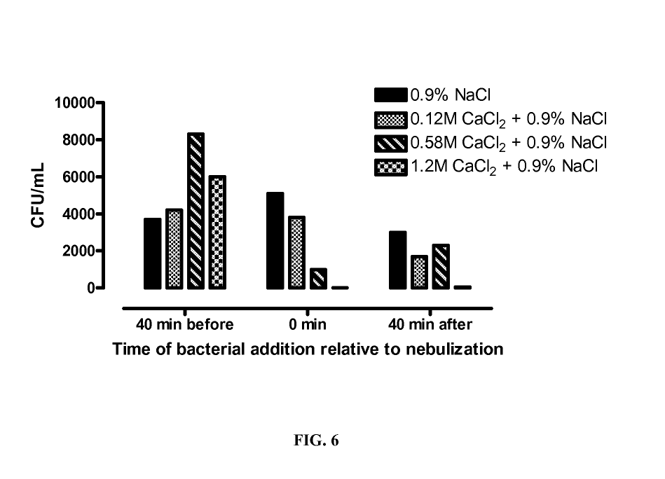

Additional studies tested whether calcium chloride formulations could influence the movement of bacteria through the mimetic when it was applied after the addition of bacteria (treatment), rather than prophylactically. Sodium alginate mimetic was exposed to a single dose of three different calcium chloride formulations: 0.12M calcium chloride in 0.90% sodium chloride, 0.58M calcium chloride in 0.90% sodium chloride and 1.2M calcium chloride in 0.90% sodium chloride. Notably, the amount of calcium delivered with these formulations in a single dose is comparable to the doses of calcium delivered in the above experiments with 0.12M calcium chloride in 0.9% sodium chloride. K. pneumoniae was added to the apical surface of the mimetic either 40 minutes before, immediately after, or 40 minutes after the exposure to formulation and the titer of bacteria recovered from the basolateral chamber after 240 minutes was determined. Similar to the data presented above, when bacteria were added to the mimetic immediately after the addition of formulation, a reduction in titer was observed relative to the saline treated control for both the 0.58M calcium chloride in 0.90% sodium chloride and 1.2M calcium chloride in 0.90% sodium chloride treatments (FIG. 6). A similar reduction was seen when bacteria were added 40 minutes after exposure to the high concentration of calcium chloride (1.2M calcium chloride). In contrast, none of the formulations tested reduced the movement of bacteria through the mimetic when bacteria were added 40 minutes before formulation exposure. These data support the idea that the addition of calcium chloride to the sodium alginate mimetic results in a surface effect that acts as a barrier that prevents the entry of bacteria into the mimetic.

1E. Calcium Chloride Inhibits the Movement of Bacteria Through Mucus Mimetic in a Dose Dependent Manner

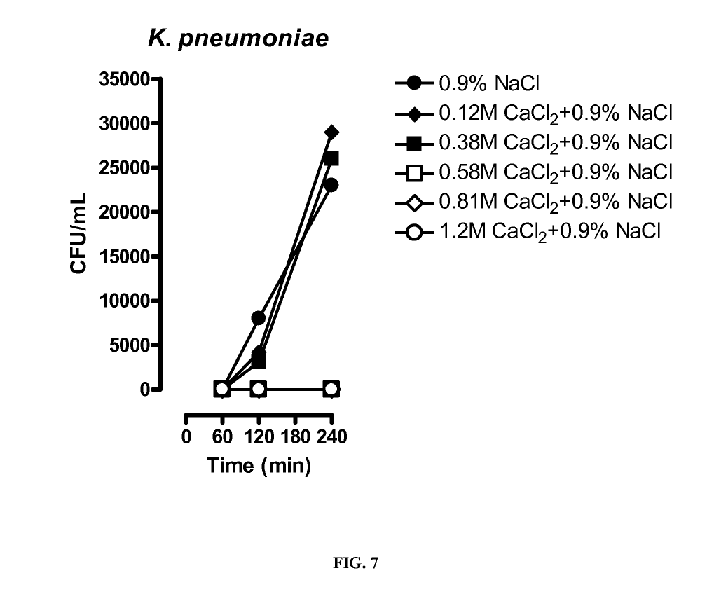

Previous data demonstrated that formulations consisting of calcium chloride at different concentrations could effectively reduce bacterial movement through sodium alginate mimetic. In these studies, the number of exposures via nebulization was different, making direct comparisons difficult. As such, the following calcium chloride formulations were tested: 0.12M calcium chloride in 0.90% sodium chloride, 0.380M calcium chloride in 0.90% sodium chloride, 0.58M calcium chloride in 0.90% sodium chloride, 0.81M calcium chloride in 0.90% sodium chloride and 1.2M calcium chloride in 0.90% sodium chloride. In this study, a single dose of each formulation was delivered to the apical surface of sodium alginate mimetic and bacteria were added immediately after exposure. Exposure to formulations with 0.58M calcium chloride or greater significantly reduced the movement across the mimetic to the limit of detection for the assay. In contrast, the 0.12M and 0.38M treatments had no effect. These data (FIG. 7) show that the critical concentration of calcium needed for the inhibition of bacteria lies between the amount delivered by the 0.38M and 0.58M solutions. From other analyses, this is approximately 3-4 μg of calcium per cm² and we hypothesize that delivery of this amount of calcium or more with any calcium containing formulation would have similar effects as that observed for calcium chloride.

1F. Calcium Chloride Alone Inhibits the Movement of Bacteria Through Mucus Mimetic in a Dose Dependent Manner

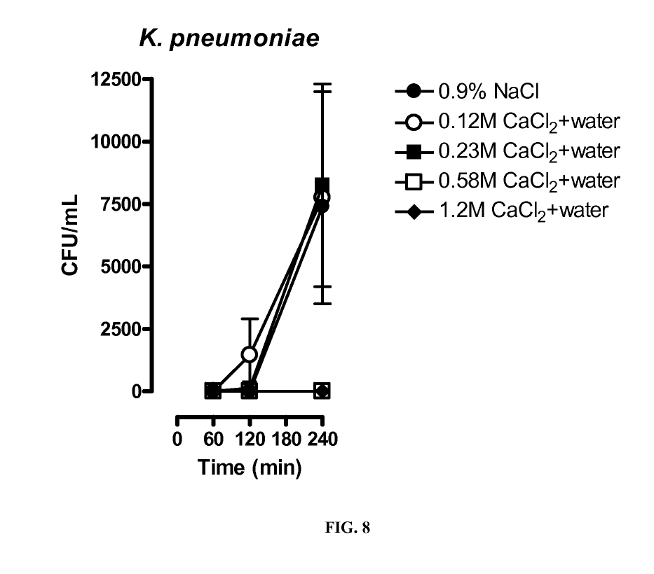

All of the formulations tested above were formulated in isotonic saline. Thus, the inhibitory effects seen could be caused by the dual action of sodium and calcium ions in concert or alternatively, the effect could be driven entirely by calcium. To differentiate between these hypotheses, we tested additional calcium formulations based in water rather than saline. The specific formulations tested were: 0.12M calcium chloride in water, 0.23M calcium chloride in water, 0.58M calcium chloride in water, and 1.2M calcium chloride in water. Similar to the data obtained with saline based formulations, delivery of both the 0.58M and 1.2M solutions reduced the movement of bacteria across the mimetic to below the limit of detection, whereas the 0.12M and 0.23M formulations had no effect compared to the control (FIG. 8). Coupled with the findings above, this shows that the inhibitory effect of calcium containing formulations is a result of calcium ion interactions with the mimetic and not due to combined effects of multiple ions.

1G. Calcium Chloride Reduces the Movement of P. aeruginosa Across Sodium Alginate Mucus Mimetic

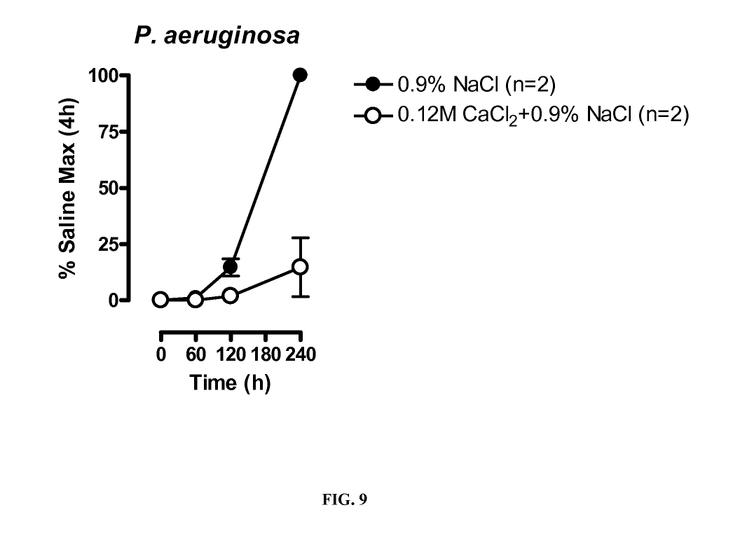

Previous work has demonstrated that exposure of sodium alginate mucus mimetic to calcium chloride reduced the movement of Klebsiella pneumoniae and a non-mucoid strain of S. pneumoniae across the mucus layer. The movement of P. aeruginosa, a gram-negative opportunistic pathogen that is a frequent cause of infection in patients with cystic fibrosis, was tested to further characterize the broad-spectrum nature of this effect. Sodium alginate was exposed to different concentrations of calcium chloride in isotonic saline: 0.12M CaCl₂ in 0.90% sodium chloride and P. aeruginosa was added to the apical surface of the mimetic. Treatment with calcium chloride significantly reduced the movement of P. aeruginosa (FIG. 9; 14.7±13% of the saline control at 4 h; n=2). Together with previous data demonstrating that the exposure of sodium alginate mimetic to formulations containing CaCl₂ in NaCl can inhibit the movement of K. pneumoniae and S. pneumoniae in this assay, these data further support the broad-spectrum nature of the treatment.

1H. Calcium Chloride Reduces the Movement of S. aureus and Non-Typeable Haemophilus influenzae (NTHI) Across Sodium Alginate Mucus Mimetic

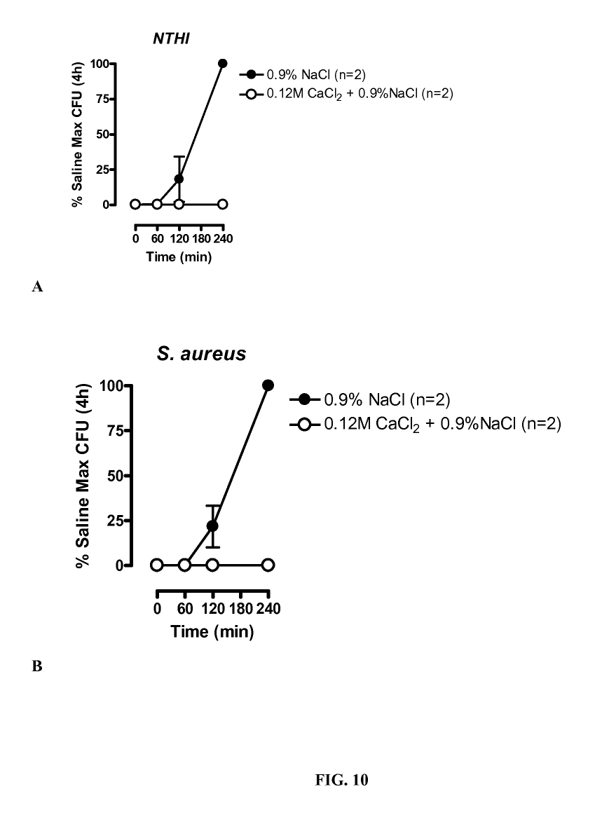

Previous work has demonstrated that exposure of sodium alginate mucus mimetic to calcium chloride reduced the movement of Klebsiella pneumoniae, S. pneumoniae, and P. aeruginosa across the mucus layer. The movement of S. aureus, a gram-positive pathogen that is a frequent cause of pneumonia, and non-typeable H. influenzae (NTHI), a gram-negative pathogen that is a cause of pneumonia and associated with exacerbations in compromised patients, were tested to further characterize the broad-spectrum nature of this effect. Sodium alginate was exposed to different concentrations of calcium chloride in isotonic saline: 0.12M CaCl2 in 0.90% sodium chloride and S. aureus or NTHI was added to the apical surface of the mimetic. Treatment with calcium chloride completely blocked the movement of NTHI (FIG. 10A); 0.3% of the saline control at 4 h; n=2) and S. aureus (FIG. 10B; 0.06% of the saline control at 4 h; n=2). Together with previous data demonstrating that the exposure of sodium alginate mimetic to formulations containing CaCl2 in NaCl can inhibit the movement of other pathogens in this assay, these data further support the broad-spectrum nature of the treatment.

1I. Calcium Chloride Reduces Formation of Particles that Contain Pathogen

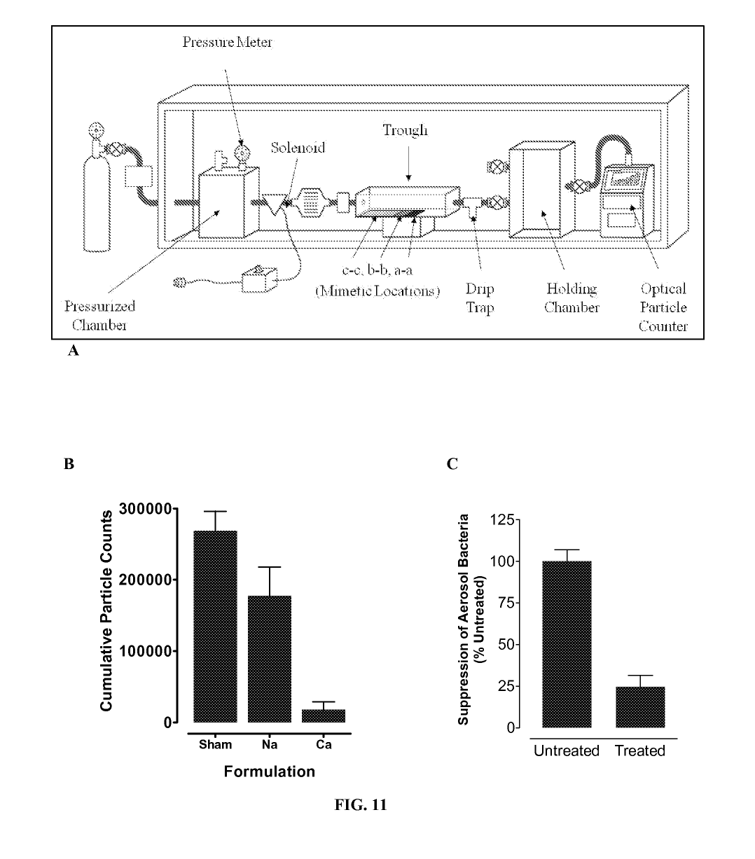

To test whether changes in the surface viscoelastic properties of mucus mimetic could translate into differences in particle formation, and thus impact transmission, studies using a simulated cough system were conducted. G. Zayas, J. Dimitry, A. Zayas, D. O'Brien, M. King, BMC Pulm Med 5, 11 (2005). The simulated cough system involves passing air, at a defined pressure, through a pneumotachograph and across a model trachea that has been lined with mucus mimetic A schematic of the system is shown in FIG. 11A. The air pressure passed through the system is such that it will mimic the flow profile and volume of a cough. To test the effect of different aerosols on particle formation, saline or calcium aerosols were topically delivered to the surface of a mucus mimetic (locus bean gum) followed by simulating a cough through the system and collecting the particles with an optical particle counter (CI-500B Climet Instruments, Redlands, Calif.). Exposure of the mimetic to 0.12M CaCl₂ in 0.90% sodium chloride reduced the number of particles relative to the control condition by 93% (FIG. 11B; n=4, p<0.01 one-way ANOVA), where as 0.90% sodium chloride treatment had only a modest effect (34% of control, n=4). Next, we tested whether the reduced particle counts would correlate with a reduction in the number of aerosolized bacteria using the same system. Mucus mimetic was mixed with Klebsiella pneumoniae and was added to the model trachea of the cough system. After exposure of the mimetic to 0.12M CaCl₂ in 0.90% sodium chloride or leaving the mimetic untreated, a cough was simulated and the particles were collected in liquid broth. Bacteria (particles) collected in the broth were diluted and plated on agar plates to enumerate the number of bacteria in each condition. Exposure of the mimetic to 0.12M CaCl₂ in 0.90% sodium chloride before cough simulation suppressed the number of particles formed by 75% compared to the untreated control (FIG. 11C). These findings show that administering salt aerosols topically to mucus surfaces can act to limit airborne spread of pathogens and reduce contagion and spread of disease.

### Example 2

### In vivo Studies

Mouse studies were conducted to assess whether salt formulations are effective in treating pneumonia in vivo.

Mouse Model

Specific pathogen-free female C57BL/6 mice (6-7 weeks, 16-22g) were used in these studies. Mice were given access to food and water ad libitum. For infections, S. pneumoniae (Serotype 3; ATCC 6303) were streaked onto blood agar plates and grown at 37° C. plus 5% CO₂ overnight. Prior to infection, animals were anesthetized by intraperitoneal injection of a mixture of ketamine and xylazine. Single colonies of S. pneumoniae were resuspended in sterile saline to OD₆₀₀=0.3 and then diluted 1:4 in saline. Colloidal carbon was added to 1% and 50 μL of the resulting solution (˜1×10⁶ CFU) was instilled into the left lung of anesthetized mice to produce infection. Following infection, the bacterial titer of the inoculum was determined by serial dilution and plating on blood agar plates. After 24 hours, mice were euthanized and the bacterial burden in lungs of infected animals was determined by plating serially diluted lung homogenate on blood agar plates.

### Salt Formulation Aerosol Delivery Systems

A whole-body exposure system using a high output nebulizer was utilized to deliver salt aerosols to a pie-chamber exposure system. Each pie chamber exposure chamber was modified such that a single tube delivered aerosol to a central manifold and ultimately to one of 11 mouse holding chambers via 4 inlet ports in each chamber. The total flow through the system was 11.7 L/min and animals were exposed to cationic aerosols for 15 minutes.

### Aerosol Characterization

Particle sizing of the aerosol generated by the high output nebulizer was performed using an inhaler adaptor set-up on a Sympatec Helos particle size analyzer outfitted with an R3 lens (0.5 to 175 μM size range). The nebulizer was filled with 45 mL isotonic saline (J T Baker, Phillipsburg, N.J.) and the outlet port of the tubing connected to the nebulizer was positioned ˜1 cm from the inhaler adaptor. Each test measurement was taken for 5 seconds (Cₒₚₜ 16.5-29.31%) and the volume median diameter (MMD; x₅₀) and the geometric standard deviation were recorded for each measurement. Flow rates were determined during each test run using a pneumotachometer and a Validyne pressure transducer connected to a voltage amplifier and voltmeter. The system was calibrated such that 1CFM=1V.

Nebulizer output rates were determined by measuring the mass deposition onto collection filters. Filters were weighed immediately before collection and immediately after a 30 second collection period. Three test runs were performed using a fresh solution of isotonic saline for each measurement.

### Salt Formulation Aerosol Dosing Estimates

Estimated CaCl₂ dose levels and aerosol concentrations are shown in Table 2.

TABLE 2

| | Exposure time | Dose Level of | Aerosol Concentration |
|---|---|---|---|
| Study | (min) | Ca^{AB}(mg/kg/day) | (mg/L) |
| Mouse | 15 | 2.3 | 0.146 |
| pneumonia | | | |

^{A}Based on the formula presented below.
^{B}This estimation of achieved dose assumes 100% deposition within the respiratory tract.

Achieved dose levels to animals during the exposure period were estimated using the following formula:

$${D}_{L} = \frac{{E}_{c} \times RMV \times T}{BW}$$

D_{L}=Achieved dose levels (mg/kg/day)
E_{c}=Actual aerosol concentration delivered to the animals (mg/L air)
RMV=Respiratory Minute Volume (L/min.) according to the method of Bide et al.: RMV (L/min.)=0.499×BW (kg)^{0.809} (estimated average over exposure period)
T=Time, duration of daily exposure (min.)
BW=Mean body weight (kg).

### Mouse Treatment Study

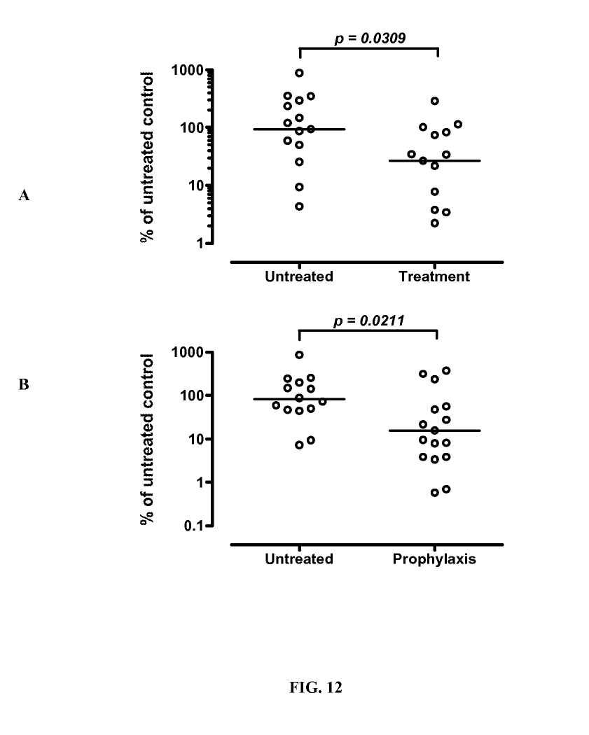

Mice were randomly assigned to different study groups on the day of the infections. Different aerosol exposure times relative to the time of infection were utilized to test the effect of aerosols in both prophylaxis and treatment regimens (FIGS. 12A and 12B). For each exposure, mice were loaded into a customized whole-body pie chamber system in which aerosols were delivered to a central manifold and subsequently to each individual animal. Aerosol exposures consisted of a 15 minute exposure of 0.12M calcium chloride in 0.90% sodium chloride, which delivered an estimated dose of 6.4 mg/kg/day of CaCl₂. After 24 hours of infection, animals were euthanized by isoflurane inhalation and the lungs were surgically removed and placed in sterile water. Lungs were homogenized using a glass mortar and pestle until no large tissue fragments were visible. Colony forming units (CFU) were enumerated by serially diluting lung homogenates in sterile water and plating on blood agar plates. Plates were incubated overnight at 37° C. plus 5% CO₂ and CFU counted the following day.

Differences between groups were evaluated by Mann-Whitney U test.

2A. Prophylactic Exposure and Treatment of Mice with Calcium Chloride Formulations Reduces the Bacterial Burden in Murine Lungs

A S. pneumoniae mouse model was employed to test the potential pathogen independence to evaluate the treatment effect of salt on a bacterial infection. As shown in FIG. 12A, treatment (2.3 mg Ca/kg deposited dose) by whole body exposure of 0.12M calcium chloride in 0.90% sodium chloride two hours post-infection led to significantly lower bacterial burden 24 hours later (n=15) relative to untreated controls (n=15). Prophylaxis in the mouse pneumonia model was evaluated by pretreating mice (n=12) with salt solutions 2 hours before installation and subsequently infecting with S. pneumoniae by intratracheal installation. FIG. 12B demonstrates that relative to untreated controls (n=12) and infected animals treated 2 hours post infection (n=15), bacterial burden 24 hours post infection was statistically lower in the prophylactic group compared to any other.

2B. Prophylactic Treatment of Bacterial Pneumonia is Driven by Calcium Chloride Specifically and not Divalent Cations in General

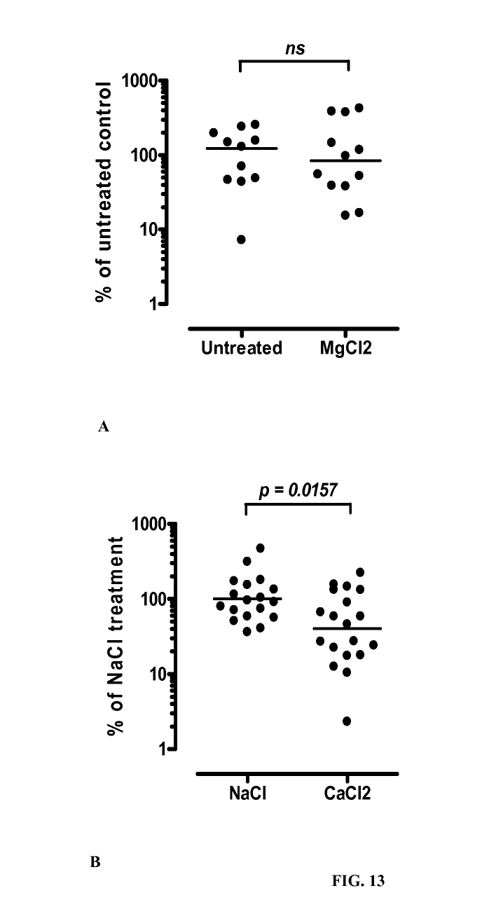

The role of the nature of the aerosolized cation was evaluated by repeating treatment studies in the mouse pneumonia model by treating animals two hours before infection with salt solutions of 2.0% magnesium chloride and 0.90% sodium chloride (n=12), as well as with 0.90% sodium chloride alone (n=12). Animals treated with the MgCl₂ formulation (FIG. 13A) and saline formulation (FIG. 13B) solutions had similar bacterial burdens as the untreated controls 24 hours post-infection, demonstrating that the efficacy of the salt formulations in treating bacterial pneumonia was specific to CaCl₂ containing formulations and not general to cations (whether monovalent or divalent).

2C. Therapeutic Activities of Calcium:Sodium Formulations in Treating Bacterial Infections

In this example, the therapeutic activities of formulations comprising calcium chloride and sodium chloride in treating bacterial infections were examined using a mouse model. The data showed that the calcium:sodium formulations were effective in treating Streptococcus pneumoniae infection in the mouse model.

Methods:

Bacteria were prepared by growing cultures on tryptic soy agar (TSA) blood plates overnight at 37° C. plus 5% CO₂. Single colonies were resuspended to an OD₆₀₀ ˜0.3 in sterile PBS and subsequently diluted 1:4 in sterile PBS [˜2×10⁷ Colony forming units (CFU)/mL]. Mice were infected with 504 of bacterial suspension (˜1×10⁶ CFU) by intratracheal instillation while under anesthesia.

C57BL6 mice were exposed to aerosolized liquid formulations in a whole-body exposure system using either a high output nebulizer or Pari LC Sprint nebulizers connected to a pie chamber cage that individually holds up to 11 animals. Treatments were performed 2 hours before infection with Serotype 3 Streptococcus pneumoniae. Unless otherwise stated, exposure times were 3 minutes in duration. Twenty-four hours after infection mice were euthanized by pentobarbital injection and lungs were collected and homogenized in sterile PBS. Lung homogenate samples were serially diluted in sterile PBS and plated on TSA blood agar plates. CFU were enumerated the following day.

Results:

(a) Calcium: Sodium Formulations (Ca²⁺:Na⁺ at 8:1 Molar Ratio) Reduced Bacterial Burden in a Dose Responsive Manner

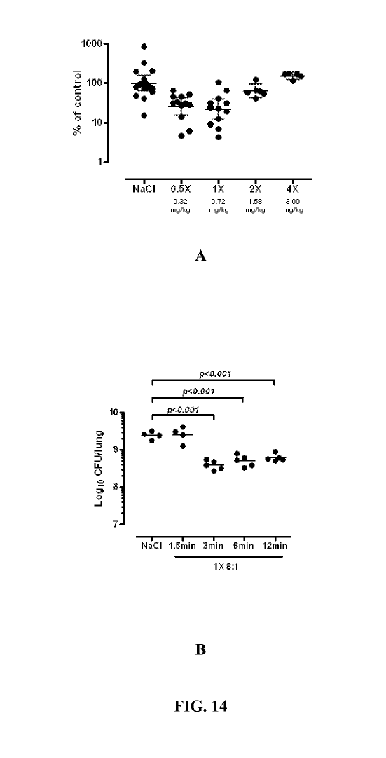

The therapeutic activities of the calcium:sodium formulations were evaluated in the same model and over a wide dose range. With dosing time held constant, different calcium doses were delivered by using formulations consisting of different concentrations of Ca²⁺:Na⁺ and therefore different tonicities. The formulations containing Ca²⁺:Na⁺ at an 8:1 molar ratio reduced bacterial burden in a dose responsive manner, with the greatest reduction observed at lower doses of calcium (about a 4-fold reduction at a dose of 0.32 mg Ca²⁺/kg and tonicity of 0.5×(Formulation 9, Table 1), and about a 5-fold reduction at a dose of 0.72 mg Ca²⁺/kg and tonicity of 1.0×) (Formulation 10, Table 1) (FIG. 14A). Interestingly, these reductions were comparable to the reduction seen for Formulation A (1.29% CaCl₂ and 0.9% NaCl), however at significantly lower doses. The 2× tonicity formulation (Formulation 11, Table 1), which is equivalent to Formulation A in tonicity, had a relatively modest effect on reducing bacterial titers (˜1.6 fold reduction) when administered at a dose of 1.58 mg Ca²⁺/kg.

(b) Increasing Dose Through Longer Nebulizations did not Significantly Affect the Therapeutic Activities of the Calcium:Sodium Formulations

FIG. 14A showed that that calcium:sodium formulations at an 8:1 ratio of calcium to sodium reduced the severity of bacterial infections at doses of less than 1.58 mg Ca²⁺/kg. Specifically, the 1× formulation (Formulation 10, ˜0.72 mg Ca²⁺/kg) was the most highly effective. The study whose results were presented in FIG. 14A tested a dose time of 3 minutes. To further examine the effect of dosage, we tested a dose range of Ca²⁺ by increasing the duration of dosing. Animals were treated with a Ca²⁺:Na⁺ formulation (1×tonicity=isotonic; 8:1 molar ratio) for different amounts of time (1.5 minutes to 12 minutes). These dose times resulted in Ca²⁺ dosages at approximately 0.36, 0.72, 1.44, and 2.88 mg Ca²⁺/kg for the 1.5, 3, 6, and 12 minutes dosing times, respectively. As shown in FIG. 14B, at the shortest dosing time, no decrease in bacterial titer was observed as compared to control animals (which were dosed 3 minutes with saline), whereas the 3, 6, and 12 minutes doses each reduced bacterial titers to statistically significant levels.

2D. Synergestic Activities of Calcium and Ampicillin in Treating Bacterial Infections

Mice (C57BL6) were exposed to nebulized solutions of Ca:Na Formulation 10 (1× tonicity; 8:1 molar ratio of Ca²⁺:Na⁺, delivered dose ˜0.72 mg Ca/kg), ampicillin in saline (96.75 mg/mL in 0.9% NaCl, delivered dose ˜3 mg/kg), or ampicillin (96.75 mg/mL) dissolved in the 1× (Formulation 10) using whole body exposure chambers. Mice were exposed to each formulation 2 h before infection with S. pneumonia. Both the 1× Ca:Na formulation and the ampicillin alone reduced bacterial burden in the lungs of infected mice to the saline control (p<0.001 Mann-Whitney U test). The 1× formulation reduced bacterial titers approximately 4.5-fold and the ampicillin reduced titers 33-fold. Unexpectedly, the combination of the two therapies resulted in an even greater reduction in bacterial titers (333-fold) than either single treatment showing a therapeutic benefit to delivering inhaled antibiotics in the calcium formulations described herein.

### Example 3

### In Vivo Mouse Model

Bacteria were prepared by growing cultures on tryptic soy agar (TSA) blood plates overnight at 37° C. plus 5% CO₂. Single colonies were resuspended to an OD₆₀₀ ˜0.3 in sterile PBS and subsequently diluted 1:4 in sterile PBS (˜2×10⁷ Colony forming units (CFU)/mL). Mice were infected with 50 μL of bacterial suspension (˜1×10⁶ CFU) by intratracheal instillation while under anesthesia.

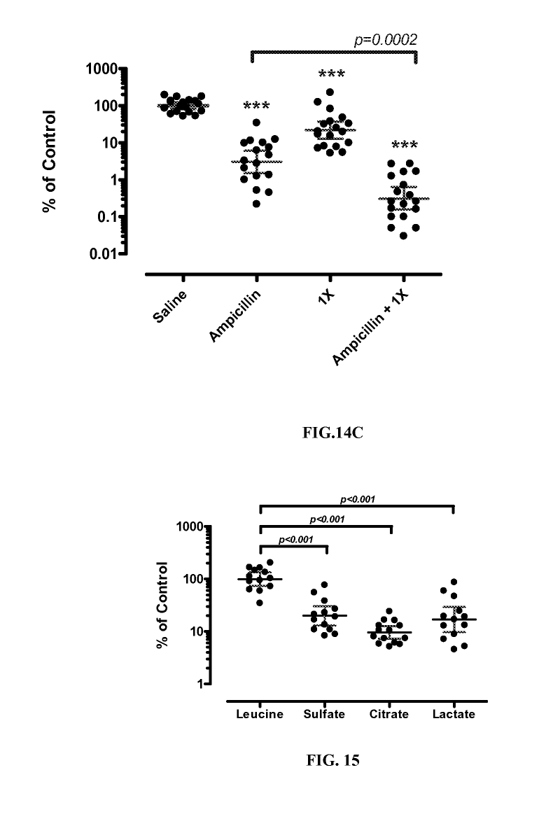

C57BL6 mice were exposed to aerosolized liquid formulations in a whole-body exposure system using either a high output nebulizer or Pari LC Sprint nebulizer connected to a pie chamber cage that individually holds up to 11 animals. Mice were treated with dry powder formulations (Table 3) 2 h before infection with S. pneumoniae. As a control, animals were exposed to a similar amount of 100% leucine dry powder. Twenty-four hours after infection mice were euthanized by pentobarbital injection and lungs were collected and homogenized in sterile PBS. Lung homogenate samples were serially diluted in sterile PBS and plated on TSA blood agar plates. CFU were enumerated the following day.
Compared to control animals, calcium dry powder treated animals exhibited reduced bacterial titers 24 hours after infection. Specifically, animals treated with a formulation comprised of calcium sulfate and sodium chloride (Formulation 3-2) exhibited 5-fold lower bacterial titers, animals treated with a formulation comprised of calcium citrate and sodium chloride (Formulation 3-1) exhibited 10.4-fold lower bacterial titers, and animals treated with a formulation comprised of calcium lactate and sodium chloride (Formulation 3-3) exhibited 5.9-fold lower bacterial titers. (FIG. 15) These data that dry powder formulations with equivalent or superior efficacy to liquid formulations can be manufactured to broadly treat bacterial and viral infections.

TABLE 3
Formulations used to evaluate efficacy

| | | Ca:Na |
|---|---|---|
| | | molar |
| Formulation | Composition | ratio |
| 3-1 | 10.0% leucine, 35.1% calcium chloride, 54.9% | 1:2 |
| | sodium citrate (Active with 12.7% calcium ion) | |
| 3-2 | 10.0% leucine, 39.6% calcium chloride, 50.4% | 1:2 |
| | sodium sulfate (Active with 14.3% calcium ion) | |
| 3-3 | 10.0% leucine, 58.6% calcium lactate, 31.4% | 1:2 |
| | sodium chloride (Active with 10.8% calcium ion) | |

While this invention has been particularly shown and described with references to preferred embodiments thereof, it will be understood by those skilled in the art that various changes in form and details may be made therein without departing from the scope of the invention encompassed by the appended claims.

1. A method for treating pneumonia, comprising administering to an individual having pneumonia an effective amount of a calcium salt formulation, wherein said calcium salt formulation is administered as an aerosol to the lungs of said individual.

2. The method of claim 1, wherein said pneumonia is bacterial pneumonia.

3. The method of claim 2, wherein said bacterial pneumonia is caused by a pathogen selected from the group consisting of Streptococcus pneumoniae, Staphylococcus aureus, Staphylococcus spp., Streptococcus spp., Streptococcus agalactiae, Haemophilus influenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Enterobacter spp., Acinetobacter spp., Acinetobacter baumannii, methicillin-resistant Staphylococcus aureus, Burkholderia spp., Stenotrophomonas maltophilia and combinations thereof.

4. The method of claim 3, wherein said bacterial pneumonia is caused by Streptococcus pneumoniae.

5. The method of claim 1, wherein said pneumonia is selected from the group consisting of community acquired pneumonia (CAP), ventilator associated pneumonia (VAP), hospital acquired pneumonia (HAP), and healthcare associated pneumonia (HCAP).

6. The method of claim 1, wherein the calcium salt is selected from the group consisting of calcium chloride, calcium carbonate, calcium acetate, calcium phosphate, calcium alginate, calcium stearate, calcium sorbate, calcium sulfate, calcium citrate, calcium lactate, and calcium gluconate.

7. The method of claim 1, wherein a calcium dose of about 0.01 mg/kg body weight to about 10 mg/kg body weight is administered to the lungs.

8. The method of claim 7, wherein the formulation is a liquid formulation or a dry powder.

9. The method of claim 1, wherein the calcium salt formulation further comprises a sodium salt.

10. The method of claim 9, wherein the sodium salt is selected from the group consisting of sodium chloride, sodium acetate, sodium bicarbonate, sodium carbonate, sodium sulfate, sodium stearate, sodium ascorbate, sodium benzoate, sodium biphosphate, sodium phosphate, sodium bisulfate, sodium citrate, sodium lactate, sodium borate, sodium gluconate, and sodium metasilicate.

11. The method of claim 10, wherein the ratio of calcium to sodium in the calcium salt formulation is about 8:1.

12. The method of claim 11, wherein a sodium dose of about 0.001 mg/kg body weight to about 10 mg/kg body weight is administered to the lungs.

13. A method for reducing transmission of pathogen which causes pneumonia, comprising administering to an individual having pneumonia, exhibiting pneumonia-like symptoms or at risk for contracting pneumonia, an effective amount of a calcium salt formulation, wherein said calcium salt formulation is administered as an aerosol to the lung of said individual.

14-17. (canceled)

18. A method of preventing pneumonia, comprising administering to an individual at risk for contracting pneumonia an effective amount of a calcium salt formulation, wherein said calcium salt formulation is administered as an aerosol to the lung of said individual.

19-21. (canceled)

22. The method of claim 18, wherein said pneumonia is selected from the group consisting of community acquired pneumonia (CAP), ventilator associated pneumonia (VAP), hospital acquired pneumonia (HAP) and healthcare associated pneumonia (HCAP).

23-41. (canceled)

42. A method for prophylaxis of ventilator-associated tracheobronchitis (VAT), comprising administering to an individual at risk for VAT an effective amount of a calcium salt formulation, wherein said calcium salt formulation is administered as an aerosol to the lung of said individual.

43-49. (canceled)
